(19) United States
(12) Patent Application Publication
(10) Pub. No.: US 2009/0306020 A1
(43) Pub. Date: Dec. 10, 2009

(54) Combination therapy comprising diaryl ureas for treating diseases

(75) Inventors: Urban Scheuring, Siegburg (DE); Ingo Bernard, Lindlar (DE); Claus Garbe, Tubingen (DE); Birgit Schittek, Bodelshusen (DE); Friedegund Meier, Tubingen (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: Bayer Healthcare AG, Leverkusen (DE)

(21) Appl. No.: 11/920,952

(22) PCT Filed: May 13, 2006
(86) PCT No.: PCT/EP2006/004523

§ 371 Date: Apr. 22, 2009

(30) Foreign Application Priority Data
May 27, 2005 (EP) 05011475.0
May 27, 2005 (EP) 05011476.8
May 27, 2005 (EP) 05011478.4

(51) Int. Cl. A61K 31/4412; C07D 213/68; A61K 31/661; A61K 31/436; A61K 31/437; A61P 35/00

(52) U.S. Cl. 514/121; 546/291; 514/346; 514/291; 514/303

(57) ABSTRACT

The present invention relates to pharmaceutical compositions and combinations for treating cancer, comprising a diaryl urea compound and an PI3K/AKT signaling pathway inhibitor. Useful combinations include e.g. BAY-43-9006 as a diaryl urea compound.

### BACKGROUND OF THE INVENTION

BAY 43-9006 refers to 4{4-[3-(4-chloro-3-trifluoromethylphenyl)-ureido]-phenoxy}-pyridine-2-carboxylic acid methyl amide and is species of diaryl urea compounds which are potent anti-cancer and anti-angiogenic agents that possess various activities, including inhibitory activity on the VEGFR, PDGFR, raf, p38, and/or flt-3 kinase signaling molecules. See, e.g., US 20050038080. The RAS/RAF/MEK/ERK pathway is involved in cellular proliferation, differentiation, and transformation, and is implicated in many cancers. The PI3K/AKT signaling pathway is another important physiological pathway in cells. It mediates extracellular stimuli, including growth factors, cytokines, cell-cell adhesion and cell-extracellular matrices (Vivanco and Sawyers, Nat Rev Cancer, 2: 489-501, 2002, Downward, Curr Opin Cell Biol, 10: 262-267, 1998). The AKT pathway appears to be active in many types of human cancer (Nicholson and Anderson, Cell Signal, 14: 381-395, 2002).

### BRIEF DESCRIPTION OF THE FIGURES

FIG. 1 (A-B). In contrast to blockade of the MAP kinase signaling pathway, blockade of the AKT signaling pathway does not affect proliferation of melanoma cells in monolayer culture. When comparing monolayer cultures from control 451Lu metastatic melanoma cells and 451Lu metastatic melanoma cells treated with the PI3K inhibitor wortmannin at dosages ranging from 2-20 μM (A), respectively, no significant effect on the number of proliferating cells was seen. In contrast, treatment of 451Lu melanoma cells with BAY 43-9006 at dosages ranging from 1-7 μM (B) resulted in a significant decrease in cell proliferation. The intensity of fluorescence, given as mean values, indicates the number of vital cells in the wells.

FIG. 2 (A-B). Blockade of AKT or MAPK signaling pathways downregulates the expression of the adhesion molecules MelCAM and avβ3 integrin, respectively, of 451Lu melanoma cells in monolayer. Monolayer cultures of 451Lu metastatic melanoma cells were treated with vehicle only, 4 μM wortmannin, 6 μM BAY 43-9006 or 4 μM wortmannin combined with 6 μM BAY 43-9006 for 96 hours, stained with antibodies against av.β3 or MelCAM, and subjected to flow cytometry. Treatment with wortmannin alone or in combination with BAY 43-9006 down-regulates cell surface expression of MelCAM (A). Cell surface expression of avβ3 integrin is down-regulated by BAY 43-9006 alone or in combination with wortmannin but not by wortmannin alone (B).

FIG. 3 (A-D). Blockade of MAPK but not of AKT signaling pathway inhibits proliferation in organotypic culture. Skmel28 metastatic melanoma cells incorporated into dermal reconstructs were treated with culture medium or culture medium with the addition of DMSO as controls, 4 μM PI3K inhibitor wortmannin, 6 μM RAF kinase inhibitor BAY 43-9006 or a combination of 4 μM wortmannin and 6 μM BAY 43-9006 and stained for Ki-67 proliferation marker (Ki-67: red, ×100). The majority of the control metastatic melanoma cells stained for Ki-67 proliferation marker (A). Treatment with the PI3K inhibitor wortmannin alone yielded little or no effect on proliferation rate (B). Treatment with BAY 43-9006 resulted in a significant decrease in cell proliferation (C). After treatment with the inhibitors in combination, no proliferating cells were detected at all (D).

FIG. 4 (A-D). Blockade of AKT and MAPK signaling pathways induces apoptosis. To investigate the pro-apoptotic effect of the PI3K inhibitor wortmannin and/or BAY 43-9006 on melanoma cells in a physiological context, control and inhibitor-treated Skmel28 metastatic melanoma reconstructs were stained for active caspase 3 (active caspase 3: red, ×50). Most of the control Skmel28 metastatic melanoma cells incorporated into dermal reconstructs were negative for active caspase 3 (A). After application of PI3K inhibitor wortmannin (B) or RAF kinase inhibitor BAY 43-9006 (C) or both (D), active caspase 3 was found in the majority of Skmel28 metastatic melanoma cells in human dermal reconstructs.

FIG. 5 (A-H). Blockade of AKT and MAPK signaling pathways downregulates the expression of the adhesion molecules MelCAM and β3 integrin, respectively. Metastatic melanoma reconstructs were treated with 4 μM PI3K inhibitor wortmannin or 6 μM BAY 43-9006 or 4 μM wortmannin combined with 6 μM BAY 43-9006 and stained for the adhesion molecules MelCAM and β3 integrin, respectively MelCAM: red, ×100; β3 integrin: red, ×50). Control metastatic melanoma cells incorporated into dermal reconstructs strongly expressed the adhesion molecules MelCAM (A) and β3 integrin (E). Blockade of AKT signaling pathway by wortmannin downregulated the expression of MelCAM (B) while blockade of MAPK signaling pathway by BAY 43-9006 did not appear to affect MelCAM expression (C) suggesting that the effect observed with the combination of both inhibitors (D) is mainly due to the blockade of the AKT pathway. The expression of β33 integrin was not altered by wortmannin treatment (F) whereas application of BAY 43-9006 alone (G) or in combination with wortmannin substantially reduced β3 integrin expression (H).

FIG. 6 (A-D). Blockade of PI3K/AKT (AKT) and RAS/RAF/MEK/ERK (MAPK) signaling pathways inhibits invasive melanoma growth in human dermal reconstructs. Skmel28 metastatic melanoma cells were incorporated into human dermal reconstructs and treated with culture medium or culture medium with the addition of DMSO as controls, 4 μM PI3K inhibitor wortmannin, 6 μM BAY 43-9006 or a combination of 4 μM wortmannin and 6 μM BAY 43-9006 and stained with hematoxylin (HE, ×100). (A) Control Skmel28 metastatic melanoma cells exhibited aggressive growth of numerous tumor cell nests and tumor cell clusters throughout the entire dermis. (B) After treatment with wortmannin, number and size of melanoma cell nests were reduced, cohesion of melanoma cells was decreased, and morphology of melanoma cells was changed with melanoma cells displaying a multidendritic phenotype. (C) BAY 43-9006 also reduced number and size of melanoma cell nests with small melanoma cell nests and single melanoma cells scattered throughout the dermis. (D) Wortmannin in combination with BAY 43-9006 completely abrogated invasive melanoma growth with very few rounded melanoma cells left in the dermis.

### DESCRIPTION OF THE INVENTION

The present invention provides drug combinations, compositions, and methods for treating diseases and conditions, including, but not limited to, cell proliferative disorders (such as cancer), inflammation, immunomodulatory disorders, and conditions associated with abnormal or undesirable angiogenesis. The drug combinations comprise at least one compound of formula I and at least one second compound that is an inhibitor of the PI3K/AKT signaling pathway. The methods can comprise, e.g., administering a diaryl urea compound as described below and a PI3K/AKT signaling pathway inhibitor, pharmaceutically-acceptable salts thereof, and derivatives thereof, etc.

The phosphatidylinositol-3-kinase (PI3K) and AKT (Protein Kinase B) signaling pathway regulates a variety of biological processes including cell survival, cell proliferation, cell growth, and cell motility. Abnormalities in PI3K-AKT signaling contribute to the pathogenesis of a number of diseases and conditions, including cell proliferative disorders (such as cancer), inflammation, and immunomodulatory disorders.

Many growth and survival factors activate PI3K family members to specifically convert one lipid signaling molecule, PIP2, into another, PI(3,4,5)P3. The phosphorylated product recruits Akt family members to the inner plasma membrane, stimulating their protein kinase activity. To date, many Akt effectors involved in several biological processes have been identified. For example, the Akt kinases mediate cell survival though phosphorylation and inactivation of apoptotic machinery components. The PI3K/AKT signaling pathway includes any members or components that participate in the signal transduction cascade. These include, but are not limited to, e.g. PI3-kinase, Akt-kinase, FKBP12, mTOR (mammalian target of rapamycin; also known as FRAP, RAFT1, or RAPT1), RAPTOR (regulatory associated protein if mTOR), TSC (tuberous sclerosis complex), PTEN, (phosphatase and tens in homolog) and downstream effectors thereof. Combinations of the present invention can be used to treat and/or prevent any condition and/or diseases associated with any of the aforementioned activities.

An inhibitor of the PI3K/AKT signaling pathway is a compound that inhibits one or more members of the aforementioned signal transduction cascade. While such compounds may be referred to as pathway inhibitors, the present invention includes the use of these inhibitors to treat any of the mentioned diseases or conditions, regardless of the mechanism of action or how the therapeutic effect is achieved. Indeed, it is recognized that such compounds may have more than one target, and the initial activity recognized for a compound may not be the activity that it possesses in vivo when administered to a subject, or whereby it achieves its therapeutic efficacy. Thus, the description of a compound as a pathway or protein target (e.g., Akt or mTOR) inhibitor indicates that a compound possesses such activity, but in no way restricts a compound to having that activity when used as a therapeutic or prophylactic agent.

Examples of AKT family members include: Akt1, Akt2 (commonly over-expressed in tumors; Bellacosa et al., Int. J. Cancer, 64:280-285, 1995), and Akt3.

Examples of PI3K family members include: p 110-alpha, p110-beta, p110-delta, and p110-gamma (catalytic).

PI3K/AKT signaling pathway inhibitors include, but are not limited to, e.g., FTY720 (e.g., Lee et al., Carcinogenesis, 25(12):2397-2405, 2004), UCN-01 (e.g., Amornphimoltham et al., Clin Cancer Res., 10(12 Pt 1):4029-37, 2004);

Examples of Phosphatidylinositol-3-kinase (PI3-kinase) inhibitors include, but are not limited to, e.g., celecoxib and analogs thereof, such as OSU-03012 and OSU-03013 (e.g., Zhu et al., Cancer Res., 64(12): 4309-18, 2004);
3-deoxy-D-myo-inositol analogs (e.g., U.S. Application No. 20040192770; Meuillet et al., Oncol. Res., 14:513-27, 2004), such as PX-316;
2′-substituted 3′-deoxy-phosphatidyl-myo-inositol analogs (e.g., Tabellini et al., Br. J. Haematol., 126(4): 574-82, 2004);
fused heteroaryl derivatives (U.S. Pat. No. 6,608,056);
3-(imidazo[1,2-a]pyridin-3-yl) derivatives (e.g., U.S. Pat. Nos. 6,403,588 and 6,653,320); Ly294002 (e.g., Vlahos, et al., J. Biol., Chem., 269(7) 5241-5248, 1994);
quinazoline-4-one derivatives, such as IC486068 (e.g., U.S. Application No. 20020161014; Geng et al., Cancer Res., 64:4893-99, 2004);
3-(hetero)aryloxy substituted benzo(b)thiophene derivatives (e.g., WO 04 108715; also WO 04 108713);
viridins, including semi-synthetic viridins such as such as PX-866 (acetic acid (1S,4E,10R,11R,13S,14R)-[4-diallylaminomethylene-6-hydroxy-1-methoxymethyl-10,13-dimethyl-3,7,17-trioxo-1,3,4,7,10,11,12,13,14,15,16,17-dodecahydro-2-oxa-cyclopenta[a]phenanthren-11-yl ester) (e.g., Ihle et al., Mol Cancer Ther., 3(7):763-72, 2004; U.S. Application No. 20020037276; U.S. Pat. No. 5,726,167); and
wortmannin and derivatives thereof (e.g., U.S. Pat. Nos. 5,504,103; 5,480,906, 5,468,773; 5,441,947; 5,378,725; 3,668,222).

Examples of Akt-kinase (also known as protein kinase B) inhibitors include, but are not limited to, e.g., Akt-1-1 (inhibits Akt1) (Barnett et al., Biochem. J., 385 (Pt. 2):399-408, 2005), Akt-1-1,2 (inhibits Ak1 and 2) (Barnett et al., Biochem. J., 385 (Pt. 2):399-408, 2005), API-59CJ-Ome (e.g., Jin et al., Br. J. Cancer., 91:1808-12, 2004), 1-H-imidazo[4,5-c]pyridinyl compounds (e.g., WO05011700), indole-3-carbinol and derivatives thereof (e.g., U.S. Pat. No. 6,656,963; Sarkar and Li, J Nutr., 134(12 Suppl):3493S-3498S, 2004), perifosine (e.g., interferes with Akt membrane localization; Dasmahapatra et al., Clin. Cancer Res., 10(15):5242-52, 2004), phosphatidylinositol ether lipid analogues (e.g., Gills and Dennis, Expert. Opin. Investig. Drugs, 13:787-97, 2004), triciribine (TCN or API-2 or NCl identifier: NSC 154020; Yang et al., Cancer Res., 64:4394-9, 2004).

Examples of mTOR inhibitors include, but are not limited to, e.g.,

FKBP12 enhancer.
rapamycins and derivatives thereof, including: CCI-779 (temsirolimus), RAD001 (Everolimus; WO 9409010), TAFA93 and AP23573; rapalogs, e.g. as disclosed in WO 98/02441 and WO 01/14387, e.g. AP23573, AP23464, AP23675, or AP23841; 40-(2-hydroxyethyl)rapamycin, 40-[3-hydroxy(hydroxymethyl)methylpropanoate]-rapamycin (also called CC1779), 40-epi-(tetrazolyt)-rapamycin (also called ABT578), 32-deoxorapamycin, 16-pentynyloxy-32(S)-dihydrorapamycin, and other derivatives disclosed in WO 05005434; derivatives disclosed in U.S. Pat. No. 5,258,389, WO 94/090101, WO 92/05179, U.S. Pat. No. 5,118,677, U.S. Pat. No. 5,118,678, U.S. Pat. No. 5,100,883, U.S. Pat. No. 5,151,413, U.S. Pat. No. 5,120,842, WO 93/111130, WO 94/02136, WO 94/02485, WO 95/14023, WO 94/02136, WO 95/16691 (e.g. SAR 943), EP 509795, WO 96/41807, WO 96/41807 and U.S. Pat. No. 5,256,790;
phosphorus-containing rapamycin derivatives (e.g., WO 05016252);
4H-1-benzopyran-4-one derivatives (e.g., U.S. Provisional Application No. 60/528,340).

Examples of phosphatidylinositol-3-kinase (PI3-kinase) inhibitors of interest are wortmannin and the derivatives or analogs thereof and the pharmaceutically acceptable salts of wortmannin and its derivatives and analogs. Consequently, methods of this invention include the use of the PI3-kinase inhibitors of formula W:

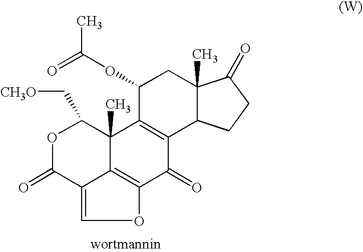

derivatives or analogs of the compound of formula W, pharmaceutically acceptable salts of the compound of formula W, and pharmaceutically acceptable salts of the derivatives or analogs of the compound of formula W.

Reference to the derivatives and analogs of wortmannin or the compound of “formula W” herein is intended to include the derivatives and analogs identified in U.S. Pat. Nos. 5,504,103; 5,480,906, 5,468,773; 5,441,947; 5,378,725; 3,668,222. Suitable derivatives and analogs of the compound of formula W include:

a) compounds of formula W1

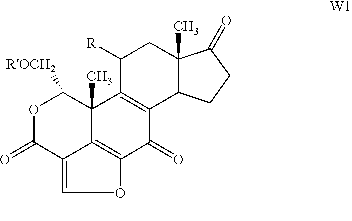

where R is H (11-desacetoxywortmannin) or acetoxy and R′ is C₁-C₆ alkyl,

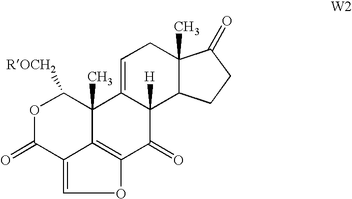

b) Δ9,11-dehydrodesacetoxywortmannin compounds of formula W2
where R′ is C₁-C₆ alkyl,
c) 17(α-dihydro-wortmannin compounds of formula W3

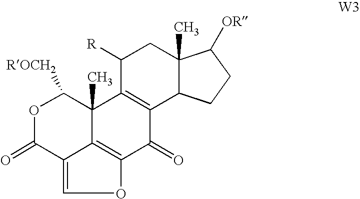

where R is H or acetoxy and R′ is C₁-C₆ alkyl, and R″ is H, C₁-C₆ alkyl,
—C(O)OH or —C(O)O—C₁-C₆ alkyl;
d) open A-ring acid or ester of wortmannin compounds of formula W4

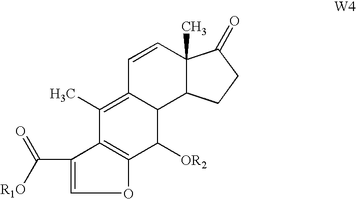

where R₁ is H, methyl or ethyl and R₂ is H or methyl or
e) 11-substituted and 17-substituted derivatives of wortmannin of formula W5

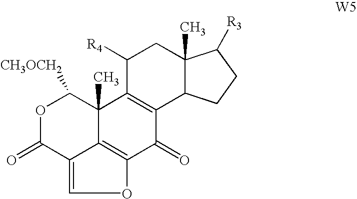

where R₄ is ═O or —O(CO)R₆, R₃ is ═O, —OH or —(CO)R₆, each R₆ is independently phenyl, C₁-C₆ alkyl or substituted C₁-C₆ alkyl, where R₄ is ═O or —OH, R₃ is not ═O.

Preference is given to PI3K inhibitors selected from celecoxib, OSU-03012, OSU-03013, PX-316, 2′-substituted, 3′-deoxy-phosphatidyl-myo-inositol derivatives, 3-imidazo[1,2-a]pyridin-3-yl) derivatives, Ly294002, IC486068, 3-(hetero)aryloxy substituted benzo(b)thiophene derivatives, PX-866, or a pharmaceutically-acceptable salts thereof. Preference is also given to mTOR inhibitors as FKBP12 enhancer and or a pharmaceutically-acceptable salts thereof.

Also preference is given to an Akt-kinase inhibitor selected from Akt-1-1, Akt-1-1,2, API-59CJ-Ome, 1-H-imidazo[4,5-c]pyridinyl derivatives, indole-3-carbinol and derivatives thereof, perifosine, phosphatidylinositol ether lipid analogues, triciribine, or a pharmaceutically acceptable salts thereof.

Also preference is given to rapamycins and derivatives thereof, including: CCI-779 (temsirolimus), RAD001 (Everolimus; WO 9409010), TAFA93 and AP23573; rapalogs, e.g. as disclosed in WO 98/02441 and WO 01/14387, e.g. AP23573, AP23464, AP23675, or AP23841; 40-(2-hydroxyethyl)rapamycin, 40-[3-hydroxy(hydroxymethyl)methylpropanoate]-rapamycin (also called CC1779), 40-epi-(tetrazolyt)-rapamycin (also called ABT578), 32-deoxorapamycin, 16-pentynyloxy-32(S)-dihydrorapamycin, and other derivatives disclosed in WO 05005434; derivatives disclosed in U.S. Pat. No. 5,258,389, WO 94/090101, WO 92/05179, U.S. Pat. No. 5,118,677, U.S. Pat. No. 5,118,678, U.S. Pat. No. 5,100,883, U.S. Pat. No. 5,151,413, U.S. Pat. No. 5,120,842, WO-93/111130, WO 94/02136, WO 94/02485, WO 95/14023, WO 94/02136, WO 95/16691 (e.g. SAR 943), EP 509795, WO 96/41807, WO 96/41807 and U.S. Pat. No. 5,256,790.

The compounds with the structure of formula (I), pharmaceutically acceptable salts, polymorphs, solvates, hydrates metabolites and prodrugs thereof, including diastereoisomeric forms (both isolated stereoisomers and mixtures of stereoisomers) are collectively referred to herein as the “compounds of formula I”.

Formula (I) is as follows:

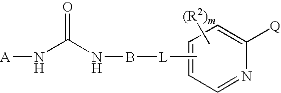

wherein
Q is —C(O)Rₓ
Rₓ is hydroxy, C₁₋₄ alkyl, C₁₋₄ alkoxy or NRₐR_{b},
Rₐ and R_{b} are independently:
a) hydrogen;
b) C₁₋₄ alkyl, optionally substituted by
   hydroxy,
   C₁₋₄ alkoxy,
   a heteroaryl group selected from pyrrole, furan, thiophene, imidazole, pyrazole, thiazole, oxazole, isoxazole, isothiazole, triazole, tetrazole, thiadiazole, oxadiazole, pyridine, pyrimidine, pyridazine, pyrazine, triazine, benzoxazole, isoquioline, quinolines and imidazopyrimidine
   a heterocyclic group selected from tetrahydropyran, tetrahydrofuran, 1,3-dioxolane, 1,4-dioxane, morpholine, thiomorpholine, piperazine, piperidine, piperidinone, tetrahydropyrimidone, pentamethylene sulfide, tetramethylene sulfide, dihydropyrane, dihydrofuran, and dihydrothiophene,
   amino, —NH₂, optionally substituted by one or two C₁₋₄ alkyl groups, or
   phenyl,
c) phenyl optionally substituted with
   halogen, or
   amino, —NH₂, optionally substituted by one or two C₁₋₄ alkyl, or
d)—a heteroaryl group selected from pyrrole, furan, thiophene, imidazole, pyrazole, thiazole, oxazole, isoxazole, isothiazole, triazole, tetrazole, thiadiazole, oxadiazole, pyridine, pyrimidine, pyridazine, pyrazine, triazine, benzoxazole, isoquioline; quinoline and imidazopyrimidine;
A is optionally substituted phenyl, pyridinyl, naphthyl, benzoxazole, isoquioline, quinoline or imidazopyrimidine;
B is optionally substituted phenyl or naphthyl:
L is a bridging group which is —S— or —O—;
M is 0, 1, 2 or 3, and
each R² is independently C₁₋₅ alkyl, C₁₋₅ haloalkyl, C₁₋₃ alkoxy, N-oxo or N-hydroxy.

Structures of optionally substituted phenyl moieties for A of formula (I) which are of particular interest include structures of formula 1xx:

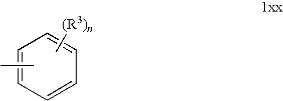

Structures of optionally substituted pyridinyl moieties for A of formula (I) which are of particular interest include structures of formula 1x:

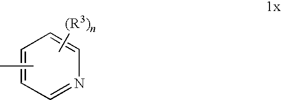

Structures of optionally substituted naphthyl moieties for A of formula (I) which are of particular interest include structures of formula 1y:

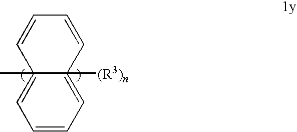

The structure 1y represents that the substituents R³ can appear on any carbon atom in either ring which has a valence that is otherwise complete with a hydrogen atom as a substituent. The bond to the urea group can also be through any carbon atom on either ring which has a valence that is otherwise complete with a hydrogen atom as a substituent.

B is optionally substituted phenyl or naphthyl. Structures of optionally substituted phenyl or naphthyl moieties for B of formula (I) which are of particular interest include structures 2a and 2b:

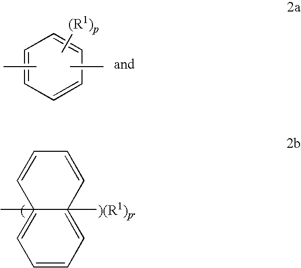

The structures 2a and 2b represent that the substituents R¹ can appear on any carbon atom in the structure which has a valence that is otherwise complete with a hydrogen atom as a substituent and the bond to the urea group can be through any carbon atom in the structure which has a valence that is otherwise complete with a hydrogen atom as a substituent.

In a class of embodiments of this invention, B is substituted by at least one halogen substituent. In another class of embodiments, Rₓ is NRₐR_{b} and Rₐ and R_{b} are independently hydrogen or C₁₋₄ alkyl optionally substituted by hydroxy and L is a bridging group which is —S— or —O—.

The variable p is 0, 1, 2, 3, or 4, typically 0 or 1. The variable n is 0, 1, 2, 3, 4, 5 or 6, typically 0, 1, 2, 3 or 4. The variable m is 0, 1, 2 or 3, typically 0.

Each R¹ is independently: halogen, C₁₋₅ haloalkyl NO₂, C(O)NR⁴R⁵, C₁₋₄ alkyl, C₁₋₆ dialkylamine, C₁₋₃ alkylamine, CN, amino, hydroxy or C₁₋₃ alkoxy. Where present, R¹ is more commonly halogen and of the halogens, typically chlorine or fluorine, and more commonly fluorine.

Each R² is independently: C₁₋₅ alkyl, C₁₋₅ haloalkyl, C₁₋₃ alkoxy, N-oxo or N-hydroxy. Where present, R² is typically methyl or trifluoromethyl.

Each R³ is independently selected from halogen, R⁴, OR⁴, S(O)R⁴, C(O)R⁴, C(O)NR⁴R⁵, oxo, cyano or nitro (NO₂).

R⁴ and R⁵ are independently selected from hydrogen, C₁₋₆ alkyl, and up to per-halogenated C₁₋₆ alkyl.

Other examples of A include: 3-tert butyl phenyl, 5-tert butyl-2-methoxyphenyl,

5-(trifluoromethyl)-2 phenyl, 3-trifluoromethyl) 4 chlorophenyl, 3-trifluoromethyl)-4-bromophenyl and 5-trifluoromethyl)-4-chloro-2 methoxyphenyl.

Other examples of B include:

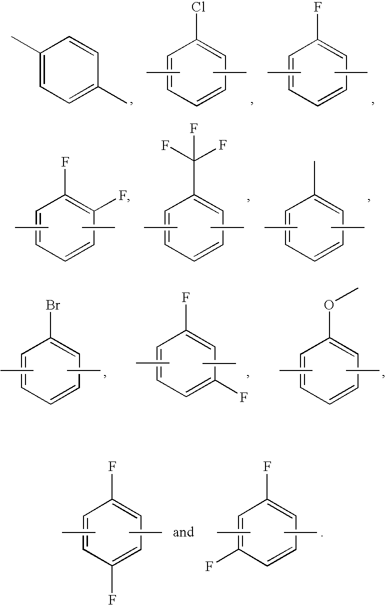

Preferably the urea group —NH—C(O)NH— and the bridging group, L, are not bound to contiguous ring carbons of B, but rather have 1 or 2 ring carbons separating them.

Examples of R¹ groups include fluorine, chorine, bromine, methyl, NO₂, C(O)NH₂, methoxy, SCH₃, trifluoromethyl, and methanesulfonyl.

Examples of R² groups include methyl, ethyl, propyl, oxygen, and cyano.

Examples of R³ groups include trifluoromethyl, methyl, ethyl, propyl, butyl, isopropyl, tert-butyl, chlorine, fluorine, bromine, cyano, methoxy, acetyl, trifluoromethanesulfonyl, trifluoromethoxy, and trifluoromethylthio.

A class of compounds of interest are of formula II below

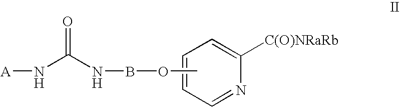

wherein Ra and Rb are independently hydrogen and C₁-C₄ alkyl,

B of formula II is

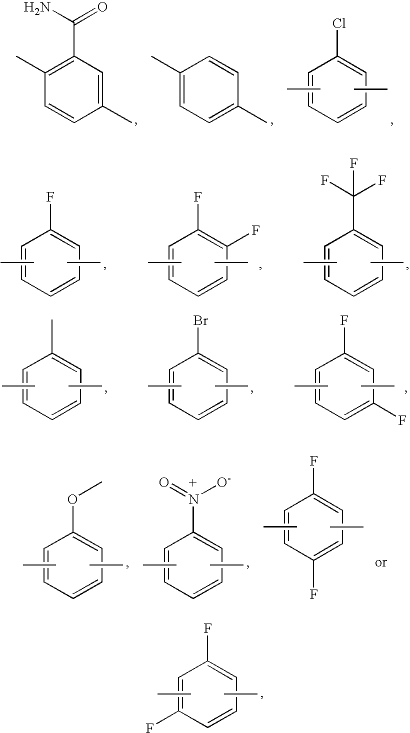

wherein the urea group, —NH—C(O)—NH—, and the oxygen bridging group are not bound to contiguous ring carbons of B, but rather have 1 or 2 ring carbons separating them,
and A of formula (II) is

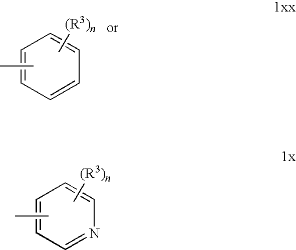

wherein the variable n is 0, 1, 2, 3 or 4.
R³ is trifluoromethyl, methyl, ethyl, propyl, butyl, isopropyl, tert-butyl, chlorine, fluorine, bromine, cyano, methoxy, acetyl, trifluoromethanesulfonyl, trifluoromethoxy, or trifluoromethylthio.

In a subclass of such compounds, each R³ substituent on A of formula II is selected from chlorine, trifluoromethyl, tert-butyl or methoxy.

In another subclass of such compounds, A of formula II is

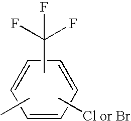

and B of formula II is phenylene, fluoro substituted phenylene or difluoro substituted phenylene.

Another class of compounds of interest includes compounds having the structure of formulae X below wherein phenyl ring “B” optionally has one halogen substituent.

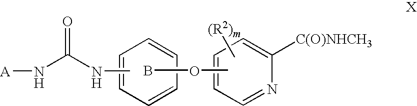

For the compounds of formula X, R², m and A are as defined above for formula I. The variable “m” is preferably zero, leaving C(O)NHCH₃ as the only substituent on the pyridinyl moiety. Preferred values for A are substituted phenyl which have at least one substituent, R³. R³ is preferably halogen, preferably Cl or F, trifluoromethyl and/or methoxy.

A subclass of compounds of interest includes compounds having the structure of formulas Z1 and Z2 below:

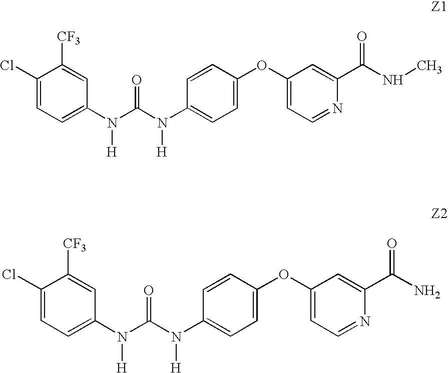

Preferably used as compound of formula I according to the invention is 4{4-[3-(4-chloro-3-trifluoromethylphenyl)-ureido]-phenoxy}-pyridine-2-carboxylic acid methyl amide (BAY 43-9006) or the p-toluenesulfonic acid salt of 4{4-[3-(4-chloro-3-trifluoromethylphenyl)-ureido]-phenoxy}-pyridine-2-carboxylic acid methyl amide (tosylate salt of compound (I)). More preferably the p-toluenesulfonic acid salt of 4{4-[3-(4-chloro-3-trifluoromethylphenyl)-ureido]-phenoxy}-pyridine-2-carboxylic acid methyl amide exists for at least 80% in the stable polymorph I. Most preferably the p-toluenesulfonic acid salt of 4{4-[3-(4-chloro-3-trifluoromethylphenyl)-ureido]-phenoxy}-pyridine-2-carboxylic acid methyl amide exists for at least 80% in the stable polymorph I and in a micronized form.

Micronization can be achieved by standard milling methods, preferably by air chat milling, known to a skilled person. The micronized form can have a mean particle size of from 0.5 to 10 μm, preferably from 1 to 6 μm, more preferably from 1 to 3 μm. The indicated particle size is the mean of the particle size distribution measured by laser diffraction known to a skilled person (measuring device: HELOS, Sympatec).

The process for preparing the p-toluenesulfonic acid salt of 4{4-[3-(4-chloro-3-trifluoromethylphenyl)-ureido]-phenoxy}-pyridine-2-carboxylic acid methyl amide and its stable polymorph I are described in the patent applications EP 04023131.8 and EP 04023130.0.

When any moiety is “substituted”, it can have up to the highest number of indicated substituents and each substituent can be located at any available position on the moiety and can be attached through any available atom on the substituent. “Any available position” means any position on the moiety that is chemically accessible through means known in the art or taught herein and that does not create an unstable molecule, e.g., incapable of administration to a human. When there are two or more substituents on any moiety, each substituent is defined independently of any other substituent and can, accordingly, be the same or different.

The term “optionally substituted” means that the moiety so modified may be either unsubstituted, or substituted with the identified substituent(s).

It is understood that the term “hydroxy” as a pyridine substituent includes 2-, 3-, and 4-hydroxypyridine, and also includes those structures referred to in the art as 1-oxo-pyridine, 1-hydroxy-pyridine or pyridine N-oxide.

Where the plural form of the word compounds, salts, and the like, is used herein, this is taken to mean also a single compound, salt, or the like.

The term C₁₋₆ alkyl, unless indicated otherwise, means straight, branched chain or cyclic alkyl groups having from one to six carbon atoms, which may be cyclic, linear or branched with single or multiple branching. Such groups include for example methyl, ethyl, n-propyl, isopropyl, n-butyl, isobutyl, sec-butyl, tert-butyl, cyclopropyl, cyclobutyl and the like.

The term C₁₋₆ haloalkyl, unless indicated otherwise, means a saturated hydrocarbon radical having up to six carbon atoms, which is substituted with a least one halogen atom, up to perhalo. The radical may be cyclic, linear or branched with single or multiple branching. The halo substituent(s) include fluoro, chloro, bromo, or iodo. Fluoro, chloro and bromo are preferred, and fluoro and chloro are more preferred. The halogen substituent(s) can be located on any available carbon. When more than one halogen substituent is present on this moiety, they may be the same or different. Examples of such halogenated alkyl substituents include but are not limited to chloromethyl, dichloromethyl, trichloromethyl, fluoromethyl, difluoromethyl, trifluoromethyl, 2,2,2-trifluoroethyl, and 1,1,2,2-tetrafluoroethyl, and the like.

The term C₁₋₆ alkoxy, unless indicated otherwise, means a cyclic, straight or branched chain alkoxy group having from one to six saturated carbon atoms which may be cyclic, linear or branched with single or multiple branching, and includes such groups as methoxy, ethoxy, n-propoxy, isopropoxy, butoxy, pentoxy and the like. It also includes halogenated groups such as 2,2-dichloroethoxy, trifluoromethoxy, and the like.

Halo or halogen means fluoro, chloro, bromo, or iodo. Fluoro, chloro and bromo are preferred, and fluoro and chloro are more preferred.

C₁₋₃alkylamine, unless indicated otherwise, means methylamino, ethylamino, propylamino or isopropylamino.

Examples of C₁₋₆ dialkylamine include but are not limited to diethylamino, ethyl-isopropylamino, methyl-isobutylamino and dihexylamino.

The term heteroaryl refers to both monocyclic and bicyclic heteroaryl rings. Monocyclic heteroaryl means an aromatic monocyclic ring having 5 to 6 ring atoms and 1-4 hetero atoms selected from N, O and S, the remaining atoms being carbon. When more than one hetero atom is present in the moiety, they are selected independently from the other(s) so that they may be the same or different. Monocyclic heteroaryl rings include, but are not limited to pyrrole, furan, thiophene, imidazole, pyrazole, thiazole, oxazole, isoxazole, isothiazole, triazole, tetrazole, thiadiazole, oxadiazole, pyridine, pyrimidine, pyridazine, pyrazine, and triazine.

Bicyclic heteroaryl means fused bicyclic moieties where one of the rings is chosen from the monocyclic heteroaryl rings described above and the second ring is either benzene or another monocyclic heteroaryl ring described above. When both rings in the bicyclic moiety are heteroaryl rings, they may be the same or different, as long as they are chemically accessible by means known in the art. Bicyclic heteroaryl rings include synthetically accessible 5-5, 5-6, or 6-6 fused bicyclic aromatic structures including, for example but not by way of limitation, benzoxazole (fused phenyl and oxazole), quinoline (fused phenyl and pyridine), imidazopyrimidine (fused imidazole and pyrimidine), and the like.

Where indicated, the bicyclic heteroaryl moieties may be partially saturated. When partially saturated either the monocyclic heteroaryl ring as described above is fully or partially saturated, the second ring as described above is either fully or partially saturated or both rings are partially saturated.

The term “5 or 6 membered heterocyclic ring, containing at least one atom selected from oxygen, nitrogen and sulfur, which is saturated, partially saturated, or aromatic” includes, by no way of limitation, tetrahydropyran, tetrahydrofuran, 1,3-dioxolane, 1,4-dioxane, morpholine, thiomorpholine, piperazine, piperidine, piperidinone, tetrahydropyrimidone, pentamethylene sulfide, tetramethylene sulfide, dihydropyrane, dihydrofuran, dihydrothiophene, pyrrole, furan, thiophene, imidazole, pyrazole, thiazole, oxazole, isoxazole, isothiazole, triazole, pyridine, pyrimidine, pyridazine, pyrazine, triazine, and the like.

The term “C₁₋₃ alkyl-phenyl” includes, for example, 2-methylphenyl, isopropylphenyl, 3-phenylpropyl, or 2-phenyl-1-methylethyl. Substituted examples include 2-[2-chlorophenyl]ethyl, 3,4-dimethylphenylmethyl, and the like.

Unless otherwise stated or indicated, the term “aryl” includes 6-12 membered mono or bicyclic aromatic hydrocarbon groups (e.g., phenyl, naphthalene, azulene, indene group) having 0, 1, 2, 3, 4, 5 or 6 substituents.

The compounds of formula (I) may contain one or more asymmetric centers, depending upon the location and nature of the various substituents desired. Asymmetric carbon atoms may be present in the (R) or (S) configuration or (R,S) configuration. In certain instances, asymmetry may also be present due to restricted rotation about a given bond, for example, the central bond adjoining two substituted aromatic rings of the specified compounds. Substituents on a ring may also be present in either cis or trans form. It is intended that all such configurations (including enantiomers and diastereomers), are included within the scope of the present invention. Preferred compounds are those with the absolute configuration of the compound of formula (I) which produces the more desirable biological activity. Separated, pure or partially purified isomers or racemic mixtures of the compounds of this invention are also included within the scope of the present invention. The purification of said isomers and the separation of said isomeric mixtures can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallization. The optically active bases or acids are then liberated from the separated diastereomeric salts. A different process for separation of optical isomers involves the use of chiral chromatography (e.g., chiral HPLC columns), with or without conventional derivation, optimally chosen to maximize the separation of the enantiomers. Suitable chiral HPLC columns are manufactured by Diacel, e.g., Chiracel OD and Chiracel OJ among many others, all routinely selectable. Enzymatic separations, with or without derivitization, are also useful. The optically active compounds of formula I can likewise be obtained by chiral syntheses utilizing optically active starting materials.

The present invention also relates to useful forms of the compounds as disclosed herein, such as pharmaceutically acceptable salts, metabolites and prodrugs. The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19. Pharmaceutically acceptable salts include those obtained by reacting the main compound, functioning as a base, with an inorganic or organic acid to form a salt, for example, salts of hydrochloric acid, sulfuric acid, phosphoric acid, methane sulfonic acid, camphor sulfonic acid, oxalic acid, maleic acid, succinic acid and citric acid. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts are prepared by reacting the compounds of the invention with the appropriate base via a variety of known methods.

Representative salts of the compounds of this invention include the conventional non-toxic salts and the quaternary ammonium salts which are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, tartrate, thiocyanate, tosylate, trifluoromethanesulfonate, and undecanoate.

Base salts include alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups may be quaternized with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and stearyl chlorides, bromides and iodides, aryl or aralkyl halides like benzyl and phenethyl bromides and others monosubstituted aralkyl halides or polysubstituted aralkyl halides.

Solvates for the purposes of the invention are those forms of the compounds where solvent molecules form a complex in the solid state and include, but are not limited to for example ethanol and methanol. Hydrates are a specific form of solvates, where the solvent molecule is water.

Certain pharmacologically active agents can be further modified with labile functional groups that are cleaved after in vivo administration to furnish the parent active agent and the pharmacologically inactive derivatizing group. These derivatives, commonly referred to as prodrugs, can be used, for example, to alter the physicochemical properties of the active agent, to target the active agent to a specific tissue, to alter the pharmacokinetic and pharmacodynamic properties of the active agent, and to reduce undesirable side effects. Prodrugs of the invention include, e.g., the esters of appropriate compounds of this invention that are well-tolerated, pharmaceutically acceptable esters such as alkyl esters including methyl, ethyl, propyl, isopropyl, butyl, isobutyl or pentyl esters. Additional esters such as phenyl-C₁-C₅ alkyl may be used, although methyl ester is preferred.

Methods which can be used to synthesize other prodrugs are described in the following reviews on the subject, which are incorporated herein by reference for their description of these synthesis methods:
Higuchi, T.; Stella, V. eds. Prodrugs As Novel Drug Delivery Systems. ACS Symposium Series. American Chemical Society: Washington, D.C. (1975).
Roche, E. B. Design of Biopharmaceutical Properties through Prodrugs and Analogs. American Pharmaceutical Association: Washington, D.C. (1977).
Sinkula, A. A.; Yalkowsky, S. H. J Pharm Sci. 1975, 64, 181-210.
Stella, V. J.; Charman, W. N. Naringrekar, V. H. Drugs 1985, 29, 455-473.
Bundgaard, H., ed. Design of Prodrugs. Elsevier: New York (1985).
Stella, V. J.; Himmelstein, K. J. J. Med. Chem. 1980, 23, 1275-1282.
Han, H-K; Amidon, G. L. AAPS Pharmsci 2000, 2, 1-11.
Denny, W. A. Eur. J. Med. Chem. 2001, 36, 577-595.
Wermuth, C. G. in Wermuth, C. G. ed. The Practice of Medicinal Chemistry Academic Press: San Diego (1996), 697-715.
Balant, L. P.; Doelker, E. in Wolff, M. E. ed. Burgers Medicinal Chemistry And Drug Discovery John Wiley & Sons: New York (1997), 949-982.

The metabolites of the compounds of this invention include oxidized derivatives of the compounds of formula I, II, X, Z1 and Z2, wherein one or more of the nitrogens are substituted with a hydroxy group; which includes derivatives where the nitrogen atom of the pyridine group is in the oxide form, referred to in the art as 1-oxo-pyridine or has a hydroxy substituent, referred to in the art as 1-hydroxy-pyridine.

### General Preparative Methods

The particular process to be utilized in the preparation of the compounds used in this embodiment of the invention depends upon the specific compound desired. Such factors as the selection of the specific substituents play a role in the path to be followed in the preparation of the specific compounds of this invention. Those factors are readily recognized by one of ordinary skill in the art.

The compounds of the invention may be prepared by use of known chemical reactions and procedures as described in the following published international applications WO 00/42012, WO03/047579, WO 2005/009961, WO 2004/078747 and WO05/000284 and European patent applications EP 04023131.8 and EP 04023130.0.

The compounds of the invention can be made according to conventional chemical methods, and/or as disclosed below, from starting materials which are either commercially available or producible according to routine, conventional chemical methods. General methods for the preparation of the compounds are given below.

The preparation of ureas of formula (I) can be prepared from the condensation of the two arylamine fragments and in the presence of phosgene, di-phosgene, tri-phosgene, carbonyldiimidazole, or equivalents in a solvent that does not react with any of the starting materials, as described in one or more of these published. Alternatively, compounds of formula (I) can be synthesized by reacting amino compounds) with isocyanate compounds as described in one or more of the published international applications described above.

The isocyanates are commercially available or can be synthesized from heterocyclic amines according to methods commonly known to those skilled in the art [e.g. from treatment of an amine with phosgene or a phosgene equivalent such as trichloromethyl chloroformate (diphosgene), bis(trichloromethyl)carbonate (triphosgene), or N,N′-carbonyldiimidazole (CDI); or, alternatively by a Curtius-type rearrangement of an amide, or a carboxylic acid derivative, such as an ester, an acid halide or an anhydride].

Aryl amines of formulas are commercially available, or can be synthesized according to methods commonly known to those skilled in the art. Aryl amines are commonly synthesized by reduction of nitroaryls using a metal catalyst, such as Ni, Pd, or Pt, and H₂ or a hydride transfer agent, such as formate, cyclohexadiene, or a borohydride (Rylander. Hydrogenation Methods; Academic Press: London, UK (1985)). Nitroaryls may also be directly reduced using a strong hydride source, such as LiAlH₄ (Seyden-Penne. Reductions by the Alumino-and borohydrides in Organic Synthesis; VCH Publishers: New York (1991)), or using a zero valent metal, such as Fe, Sn or Ca, often in acidic media. Many methods exist for the synthesis of nitroaryls (March. Advanced Organic Chemistry, 3^{rd} Ed.; John Wiley: New York (1985). Larock. Comprehensive Organic Transformations; VCH Publishers: New York (1989)). Nitro aryls are commonly formed by electrophilic aromatic nitration using HNO₃, or an alternative NO₂⁺ source.

Pyridine-1-oxides of formula (I) where the pyridine ring carries a hydroxy substituent on its nitrogen atom, and A, B, L are broadly defined as above can be prepared from the corresponding pyridines using oxidation conditions know in the art. Some examples are as follows:
peracids such as meta chloroperbenzoic acids in chlorinated solvents such as dichloromethane, dichloroethane, or chloroform (Markgraf et al., Tetrahedron 1991, 47, 183);
(Me₃SiO)₂ in the presence of a catalytic amount of perrhenic acid in chlorinated solvents such as dichloromethane (Coperet et al., Terahedron Lett. 1998, 39, 761);
Perfluoro-cis-2-butyl-3-propyloxaziridine in several combinations of halogenated solvents (Amone et al., Tetrahedron 1998, 54, 7831);
Hypofluoric acid—cetonitrile complex in chloroform (Dayan et al., Synthesis 1999, 1427);
Oxone, in the presence of a base such as KOH, in water (Robker et al., J. Chem. Res., Synop. 1993, 10, 412);
Magnesium monoperoxyphthalate, in the presence of glacial acetic acid (Klemm et al., J. Heterocylic Chem. 1990, 6, 1537);
Hydrogen peroxide, in the presence of water and acetic acid (Lin A. J., Org. Prep. Proced Int. 1991, 23(1), 114);
Dimethyldioxirane in acetone (Boyd et al., J. Chem. Soc., Perkin Trans. 1991, 9, 2189).

In addition, specific methods for preparing diaryl ureas and intermediate compounds are already described elsewhere in the patent literature, and can be adapted to the compounds of the present invention. For example, Miller S. et al, “Inhibition of p38 Kinase using Symmetrical and Unsymmetrical Diphenyl Ureas” PCT Int. Appl. WO 99 32463, Miller, S et al. “Inhibition of raf Kinase using Symmetrical and Unsymmetrical Substituted Diphenyl Ureas” PCT Int. Appl., WO 99 32436, Dumas, J. et al., “Inhibition of p38 Kinase Activity using Substituted Heterocyclic Ureas” PCT Int. Appl., WO 99 32111, Dumas, J. et al., “Method for the Treatment of Neoplasm by Inhibition of raf Kinase using N-Heteroaryl-N-(hetero)arylureas” PCT Int. Appl., WO 99 32106, Dumas, J. et al., “Inhibition of p38 Kinase Activity using Aryl- and Heteroaryl-Substituted Heterocyclic Ureas” PCT Int. Appl., WO 99 32110, Dumas, J., et al., “Inhibition of raf Kinase using Aryl- and Heteroaryl-Substituted Heterocyclic Ureas” PCT Int. Appl., WO 99 32455, Riedl, B., et al., “O-Carboxy Aryl Substituted Diphenyl Ureas as raf Kinase Inhibitors” PCT Int. Appl., WO 00 42012, Riedl, B., et al., “O-Carboxy Aryl Substituted Diphenyl Ureas as p38 Kinase Inhibitors” PCT Int. Appl., WO 00 41698, Dumas, J. et al. “Heteroaryl ureas containing nitrogen hetero-atoms as p38 kinase inhibitors” US. Pat. Appl. Publ., US 20020065296, Dumas, J. et al. “Preparation of N-aryl-N′-[(acylphenoxy)phenyl]ureas as raf kinase inhibitors” PCT Int. Appl., WO 02 62763, Dumas, J. et al. “Inhibition of raf kinase using quinolyl, isoquinolyl or pyridyl ureas” PCT Int. Appl., WO 02 85857, Dumas, J. et al. “Preparation of quinolyl, isoquinolyl or pyridyl-ureas as inhibitors of raf kinase for the treatment of tumors and/or cancerous cell growth” US. Pat. Appl. Publ., US 20020165394. All the preceding patent applications are hereby incorporated by reference.

Synthetic transformations that may be employed in the synthesis of compounds of formula (I) and in the synthesis of intermediates involved in the synthesis of compounds of formula (I) are known by or accessible to one skilled in the art. Collections of synthetic transformations may be found in compilations, such as:
J. March. Advanced Organic Chemistry, 4^{th} ed.; John Wiley: New York (1992);
R. C. Larock. Comprehensive Organic Transformations, 2^{nd} ed.; Wiley-VCH: New York (1999);
F. A. Carey; R. J. Sundberg. Advanced Organic Chemistry, 2^{nd} ed.; Plenum Press: New York (1984);
T. W. Greene; P. G. M. Wuts. Protective Groups in Organic Synthesis, 3^{rd} ed.; John Wiley: New York (1999);
L. S. Hegedus. Transition Metals in the Synthesis of Complex Organic Molecules, 2^{nd} ed.; University Science Books: Mill Valley, Calif. (1994);
L. A. Paquette, Ed. The Encyclopedia of Reagents for Organic Synthesis; John Wiley: New York (1994);
A. R. Katritzky; O. Meth-Cohn; C. W. Rees, Eds. Comprehensive Organic Functional Group Transformations; Pergamon Press: Oxford, UK (1995);
G. Wilkinson; F. G A. Stone; E. W. Abel, Eds. Comprehensive Organometallic Chemistry; Pergamon Press: Oxford, UK (1982);
B. M. Trost; I. Fleming. Comprehensive Organic Synthesis; Pergamon Press: Oxford, UK (1991);
A. R. Katritzky; C. W. Rees Eds. Comprehensive Heterocylic Chemistry; Pergamon Press: Oxford, UK (1984);
A. R. Katritzky; C. W. Rees; E. F. V. Scriven, Eds. Comprehensive Heterocylic Chemistry II; Pergamon Press: Oxford, UK (1996); and
C. Hansch; P. G. Sammes; J. B. Taylor, Eds. Comprehensive Medicinal Chemistry: Pergamon Press: Oxford, UK (1990).

In addition, recurring reviews of synthetic methodology and related topics include Organic Reactions; John Wiley: New York; Organic Syntheses; John Wiley: New York; Reagents for Organic Synthesis: John Wiley: New York; The Total Synthesis of Natural Products; John Wiley: New York; The Organic Chemistry of Drug Synthesis; John Wiley: New York; Annual Reports in Organic Synthesis; Academic Press: San Diego Calif.; and Methoden der Organischen Chemie(Houben-Weyl); Thieme: Stuttgart, Germany. Furthermore, databases of synthetic transformations include Chemical Abstracts, which may be searched using either CAS OnLine or SciFinder, Handbuch der Organischen Chemie (Beilstein), which may be searched using SpotFire, and REACCS.

The compounds of formula I have been previously characterized as having various activities, including for inhibiting the Raf/MEK/ERK pathway, c-raf, b-raf, p38, VEGFR, VEGFR2, VEGR3, FLT3, PDGFR, PDGFR-beta, and c-kit. These activities and their use in treating various diseases and conditions are disclosed in, e.g., WO 00/42021, WO 00/41698, WO03/068228, WO 03/047579, WO 2005/009961, WO 2005/000284 and U.S. Application No. 20050038080, which are hereby incorporated by reference in their entirety.

### Indications

Drug combinations of the present invention can be utilized to treat any diseases or conditions that are associated with, or mediated by, the cellular pathways modulated by the compounds comprising the combinations. These pathways, include, but are not limited to signaling pathways which comprise, e.g., VEGFR, VEGFR2, Raf/Mek/Erk, Akt/PI3K, MTOR, PTEN, etc. (see also above). The drug combinations can be useful to treat diseases that are associated with, or mediated by, mutations in one of more genes present in these pathways, including cancer-associated mutations in PTEN, ras, Raf, Akt, PI3K, etc.

As mentioned above, although the compounds may be known as specific inhibitors, the present invention includes any ameliorative or therapeutic effect, regardless of the mechanism of action or how it is achieved.

The drug combination can have one or more of the following activities, including, anti-proliferative; anti-tumor; anti-angiogenic; inhibiting the proliferation of endothelial or tumor cells; anti-neoplastic; immunosuppressive; immunomodulatory; apoptosis-promoting, etc.

Conditions or diseases that can be treated in accordance with the present invention include proliferative disorders (such as cancer), inflammatory disorders, immuno-modulatory disorders, allergy, autoimmune diseases, (such as rheumatoid arthritis, or multiple sclerosis), abnormal or excessive angiogenesis, etc.

Any tumor or cancer can be treated, including, but not limited to, cancers having one or more mutations in raf, VEGFR-2, VEGFR-3, PDGFR-beta, Flt-3, ras, PTEN, Akt, PI3K, mTOR, as well as any upstream or downstream member of the signaling pathways of which they are a part. A tumor or cancer can be treated with a drug combination of the present invention irrespective of the mechanism that is responsible for it. Cancers of any organ can be treated, including cancers of, but are not limited to, e.g., colon, pancreas, breast, prostate, bone, liver, kidney, lung, testes, skin, pancreas, stomach, prostate, ovary, uterus, head and neck, blood cell, lymph, etc.

Cancers that can be treated in accordance with the present invention include, especially, but not limited to, brain tumors, breast cancer, bone sarcoma (e.g., osteosarcoma and Ewings sarcoma), bronchial premalignancy, endometrial cancer, glioblastoma, hematologic malignancies, hepatocellular carcinoma, Hodgkin's disease, kidney neoplasms, leukemia, leimyosarcoma, liposarcoma, lymphoma, Lhermitte-Duclose disease, malignant glioma, melanoma, malignant melanoma, metastases, multiple myeloma, myeloid metaplasia, myeloplastic syndromes, non-small cell lung cancer, pancreatic cancer, prostate cancer, renal cell carcinoma (e.g., advanced, advanced refractory), rhabdomyosarcoma, soft tissue sarcoma, squamous epithelial carcinoma of the skin, cancers associated with loss of function of PTEN; activated Akt (e.g. PTEN null tumors and tumors with ras mutations).

Examples of breast cancer include, but are not limited to, invasive ductal carcinoma, invasive lobular carcinoma, ductal carcinoma in situ, and lobular carcinoma in situ.

Examples of cancers of the respiratory tract include, but are not limited to, small-cell, non-small-cell lung carcinoma, bronchial adenoma, and pleuropulmonary blastoma.

Examples of brain cancers include, but are not limited to, brain stem and hypophtalmic glioma, cerebellar and cerebral, astrocytoma, medulloblastoma, ependymoma, and neuroectodermal and pineal tumor.

Tumors of the male reproductive organs include, but are not limited to, prostate and testicular cancer. Tumors of the female reproductive organs include, but are not limited to, endometrial, cervical, ovarian, vaginal, and vulvar cancer, as well as sarcoma of the uterus.

Tumors of the digestive tract include, but are not limited to, anal, colon, colorectal, esophageal, gallbladder, gastric, pancreatic, rectal, small intestine, and salivary gland cancers.

Tumors of the urinary tract include, but are not limited to, bladder, penile, kidney, renal pelvis, ureter, and urethral cancers.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, squamous cell carcinoma, Kaposi's sarcoma, malignant melanoma, Merkel cell skin cancer, and non-melanoma skin cancer.

Head-and-neck cancers include, but are not limited to, laryngeal, hypopharyngeal, nasopharyngeal, and/or oropharyngeal cancers, and lip and oral cavity cancer.

Lymphomas include, but are not limited to, AIDS-related lymphoma, non-Hodgkin's lymphoma, cutaneous T-cell lymphoma, Hodgkin's disease, and lymphoma of the central nervous system.

Sarcomas include, but are not limited to, sarcoma of the soft tissue, osteosarcoma, malignant fibrous histiocytoma, lymphosarcoma, and rhabdomyosarcoma.

Leukemias include, but are not limited to, acute myeloid leukemia, acute lymphoblastic leukemia, chronic lymphocytic leukemia, chronic myelogenous leukemia, and hairy cell leukemia.

In addition to inhibiting the proliferation of tumor cells, drug combination of the present invention can also cause tumor regression, e.g., a decrease in the size of a tumor, or in the extent of cancer in the body.

Preference is given to the treatment of melanoma, renal cancer, hepatocellular cancer, non small lung cancer, ovarian cancer, prostate cancer, colorectal cancer, breast cancer or pancreatic cancer.

Angiogenesis-related conditions and disorders can also be treated with drug combinations of the present invention. Inappropriate and ectopic expression of angiogenesis can be deleterious to an organism. A number of pathological conditions are associated with the growth of extraneous blood vessels. These include, e.g., diabetic retinopathy, neovascular glaucoma, psoriasis, retrolental fibroplasias, angiofibroma, inflammation, restenosis, etc. In addition, the increased blood supply associated with cancerous and neoplastic tissue, encourages growth, leading to rapid tumor enlargement and metastasis. Moreover, the growth of new blood vessels in a tumor provides an escape route for renegade cells, encouraging metastasis and the consequence spread of the cancer.

Useful systems for modulating angiogenesis, include, e.g., neovascularization of tumor explants (e.g., U.S. Pat. Nos. 5,192,744; 6,024,688), chicken chorioallantoic membrane (CAM) assay (e.g., Taylor and Folkman, Nature, 297:307-312, 1982; Eliceiri et al., J. Cell Biol., 140, 1255-1263, 1998), bovine capillary endothelial (BCE) cell assay (e.g., U.S. Pat. No. 6,024,688; Polyerini, P. J. et al., Methods Enzymol., 198: 440-450, 1991), migration assays, and HUVEC (human umbilical cord vascular endothelial cell) growth inhibition assay (e.g., U.S. Pat. No. 6,060,449). In addition, useful systems for modulating lymphangiogenesis, include, e.g., rabbit ear model (e.g., Szuba et al., FASEB J., 16(14):1985-7, 2002).

Modulation of angiogenesis can be determined by any suitable method. For example, the degree of tissue vascularity is typically determined by assessing the number and density of vessels present in a given sample. For example, microvessel density (MVD) can be estimated by counting the number of endothelial clusters in a high-power microscopic field, or detecting a marker specific for microvascular endothelium or other markers of growing or established blood vessels, such as CD31 (also known as platelet-endothelial cell adhesion molecule or PECAM). A CD31 antibody can be employed in conventional immunohistological methods to immunostain tissue sections as described by, e.g., Penfold et al., Br. J. Oral and Maxill. Surg., 34: 37-41; U.S. Pat. No. 6,017,949; Dellas et al., Gyn. Oncol., 67:27-33, 1997; and others. Other markers for angiogenesis, include, e.g., Vezf1 (e.g., Xiang et al., Dev. Bio., 206:123-141, 1999), angiopoietin, Tie-1, and Tie-2 (e.g., Sato et al., Nature, 376:70-74, 1995).

The drug combinations of this invention also have a broad therapeutic activity to treat or prevent the progression of a broad array of diseases, such as inflammatory conditions, coronary restenosis, tumor-associated angiogenesis, atherosclerosis, autoimmune diseases, inflammation, certain kidney diseases associated with proliferation of glomerular or mesangial cells, and ocular diseases associated with retinal vessel proliferation, psoriasis, hepatic cirrhosis, diabetes, atherosclerosis, restenosis, vascular graft restenosis, in-stent stenosis, angiogenesis, ocular diseases, pulmonary fibrosis, obliterative bronchiolitis, glomerular nephritis, rheumatoid arthritis.

The present invention also provides for treating, preventing, modulating, etc., one or more of the following conditions in humans and/or other mammals: retinopathy, including diabetic retinopathy, ischemic retinal-vein occlusion, retinopathy of prematurity and age related macular degeneration; rheumatoid arthritis, psoriasis, or bullous disorder associated with subepidermal blister formation, including bullous pemphigoid, erythema multiforme, or dermatitis herpetiformis, rheumatic fever, bone resorption, postmenopausal osteoperosis, sepsis, gram negative sepsis, septic shock, endotoxic shock, toxic shock syndrome, systemic inflammatory response syndrome, inflammatory bowel disease (Crohn's disease and ulcerative colitis), Jarisch-Herxheimer reaction, asthma, adult respiratory distress syndrome, acute pulmonary fibrotic disease, pulmonary sarcoidosis, allergic respiratory disease, silicosis, coal worker's pneumoconiosis, alveolar injury, hepatic failure, liver disease during acute inflammation, severe alcoholic hepatitis, malaria (Plasmodium falciparum malaria and cerebral malaria), non-insulin-dependent diabetes mellitus (NIDDM), congestive heart failure, damage following heart disease, atherosclerosis, Alzheimer's disease, acute encephalitis, brain injury, multiple sclerosis (demyelation and oligiodendrocyte loss in multiple sclerosis), advanced cancer, lymphoid malignancy, pancreatitis, impaired wound healing in infection, inflammation and cancer, myelodysplastic syndromes, systemic lupus erythematosus, biliary cirrhosis, bowel necrosis, radiation injury/toxicity following administration of monoclonal antibodies, host-versus-graft reaction (ischemia reperfusion injury and allograft rejections of kidney, liver, heart, and skin), lung allograft rejection (obliterative bronchitis), or complications due to total hip replacement, ad an infectious disease selected from tuberculosis, Helicobacter pylori infection during peptic ulcer disease, Chaga's disease resulting from Trypanosoma cruzi infection, effects of Shiga-like toxin resulting from E. coli infection, effects of enterotoxin A resulting from Staphylococcus infection, meningococcal infection, and infections from Borrelia burgdorferi, Treponema pallidum, cytomegalovirus, influenza virus, Theiler's encephalomyelitis virus, and the human immunodeficiency virus (HIV), papilloma, blastoglioma, Kaposi's sarcoma, melanoma, lung cancer, ovarian cancer, prostate cancer, squamous cell carcinoma, astrocytoma, head cancer, neck cancer, bladder cancer, breast cancer, colorectal cancer, thyroid cancer, pancreatic cancer, gastric cancer, hepatocellular carcinoma, leukemia, lymphoma, Hodgkin's disease, Burkitt's disease, arthritis, rheumatoid arthritis, diabetic retinopathy, angiogenesis, restenosis, in-stent restenosis, vascular graft restenosis, pulmonary fibrosis, hepatic cirrhosis, atherosclerosis, glomerulonophritis, diabetic nephropathy, thrombic micoangiopathy syndromes, transplant rejection, psoriasis, diabetes, wound healing, inflammation, and neurodegenerative diseases, hyperimmune disorders, hemangioma, myocardial angiogenesis, coronary and cerebral collateral vascularization, ischemia, corneal disease, rubeosis, neovascular glaucoma, macular degeneration retinopathy of prematurity, wound healing, ulcer Helicobacter related diseases, fractures, endometriosis, a diabetic condition, cat scratch fever, thyroid hyperplasia, asthma or edema following burns, trauma, chronic lung disease, stroke, polyps, cysts, synovitis, chronic and allergic inflammation, ovarian hyperstimulation syndrome, pulmonary and cerebral edema, keloid, fibrosis, cirrhosis, carpal tunnel syndrome, adult respiratory distress syndrome, ascites, an ocular condition, a cardiovascular condition, Crow-Fukase (POEMS) disease, Crohn's disease, glomerulonophritis, osteoarthritis, multiple sclerosis, graft rejection, Lyme disease, sepsis, von Hippel Lindau disease, pemphigoid, Paget's disease, polycystic kidney disease, sarcoidosis, throiditis, hyperviscosity syndrome, Osler-Weber-Rendu disease, chronic occlusive pulmonary disease, radiation, hypoxia, preeclampsia, menometrorrhagia, endometriosis, infection by Herpes simplex, ischemic retinopathy, corneal angiogenisis, Herpes Zoster, human immunodeficiency virus, parapoxvirus, protozoa, toxoplasmosis, spondylarthritis, ankylosing spondylitis, Morbus Bechterew, avian influenza including e.g. serotype H5N1, and tumor-associated effusions and edema.

The present invention provides methods of treating any of the aforementioned diseases and/or conditions (including those mentioned in any of the cited references), comprising administering effective amounts of at least one compound of formula I and at least one compound which is an AKT/PI3K signaling pathway inhibitor. An “effective amount” is the quantity of the compound that is useful to achieve the desired result, e.g., to treat the disease or condition.

The present invention also relates to methods of inhibiting angiogenesis in a system comprising cells, comprising administering to the system a combination of effective amounts of compounds described herein. A system comprising cells can be an in vivo system, such as a tumor in a patient, isolated organs, tissues, or cells, in vitro assays systems (CAM, BCE, etc), animal models (e.g., in vivo, subcutaneous, cancer models), hosts in need of treatment (e.g., hosts suffering from diseases having an angiogenic component, such as cancer; experiencing restenosis), etc.

In addition, the drug combinations can be administered to modulate one or more the following processes, cell growth (e.g., proliferation), tumor cell growth (including, e.g., differentiation, cell survival, and/or proliferation), tumor regression, endothelial cell growth (including, e.g., differentiation, cell survival, and/or proliferation), angiogenesis (blood vessel growth), angiogenesis, and/or hematopoiesis (e.g., proliferation, T-cell development, etc.).

Compounds or drug combinations of the present invention can be administered in any form by any effective route, including, e.g., oral, parenteral, enteral, intravenous, intraperitoneal, topical, transdermal (e.g., using any standard patch), ophthalmic, nasally, local, non-oral, such as aerosal, inhalation, subcutaneous, intramuscular, buccal, sublingual, rectal, vaginal, intra-arterial, and intrathecal, etc. They can be administered alone, or in combination with any ingredient(s), active or inactive. They can be administered in any effective dosage, e.g., from about 0.1 to about 200 mg/kg of total body weight.

The combinations of the present invention can be administered at any time and in any effective form. For example, the compounds can be administered simultaneously, e.g., as a single composition or dosage unit (e.g., a pill or liquid containing both compositions), or they can be administered as separate compositions, but at the same time (e.g., where one drug is administered intravenously and the other is administered orally or intramuscularly. The drugs can also be administered sequentially at different times. Agents can be formulated conventionally to achieve the desired rates of release over extended period of times, e.g., 12-hours, 24-hours. This can be achieved by using agents and/or their derivatives which have suitable metabolic half-lives, and/or by using controlled release formulations.

The drug combinations can be synergistic, e.g., where the joint action of the drugs is such that the combined effect is greater than the algebraic sum of their individual effects. Thus, reduced amounts of the drugs can be administered, e.g., reducing toxicity or other deleterious or unwanted effects, and/or using the same amounts as used when the agents are administered alone, but achieving greater efficacy, e.g., in having more potent antiproliferative and pro-apoptotic action.

Compounds or drug combinations of the present invention can be further combined with any other suitable additive or pharmaceutically acceptable carrier. Such additives include any of the substances already mentioned, as well as any of those used conventionally, such as those described in Remington: The Science and Practice of Pharmacy (Gennaro and Gennaro, eds, 20th edition, Lippincott Williams & Wilkins, 2000); Theory and Practice of Industrial Pharmacy (Lachman et al., eds., 3rd edition, Lippincott Williams & Wilkins, 1986); Encyclopedia of Pharmaceutical Technology (Swarbrick and Boylan, eds., 2nd edition, Marcel Dekker, 2002). These can be referred to herein as “pharmaceutically acceptable carriers” to indicate they are combined with the active drug and can be administered safely to a subject for therapeutic purposes.

In addition, compounds or drug combinations of the present invention can be administered with other active agents or therapies (e.g., radiation) that are utilized to treat any of the above-mentioned diseases and/or conditions.

The present invention provides combinations of at least one compound of Formula I and at least one compound selected from list A, e.g., which is a PI3K/AKT signalling pathway inhibitor useful in treating a disease or disorder. “Combinations” for the purposes of the invention include:
single compositions or dosage forms which contain at least one compound of Formula I and at least one second compound which is an PI3K/AKT signalling pathway inhibitor;
combination packs containing at least one compound of Formula I and at least one second compound which is an PI3K/AKT signalling pathway inhibitor, to be administered concurrently or sequentially;
kits which comprise at least one compound of Formula I and at least one second compound which is an PI3K/AKT signalling pathway inhibitor packaged separate from one another as unit dosages or as independent unit dosages, with or without instructions that they be administered concurrently or sequentially; and
separate independent dosage forms of at least one compound of Formula I and at least one second compound which is an PI3K/AKT signalling pathway inhibitor which cooperate to achieve a therapeutic effect, e.g., prophylaxis or treatment of the same disease, when administered concurrently or sequentially.

The dosage of each agent of the combination can be selected with reference to the other and/or the type of disease and/or the disease status in order to provide the desired therapeutic activity. For example, the active agents in the combination can be present and administered in a fixed combination. “Fixed combination” is intended here to mean pharmaceutical forms in which the components are present in a fixed ratio that provides the desired efficacy. These amounts can be determined routinely for a particular patient, where various parameters are utilized to select the appropriate dosage (e.g., type of cancer, age of patient, disease status, patient health, weight, etc.), or the amounts can be relatively standard.

The combination can comprise effective amounts of at least one compound of Formula I and at least one second compound which is a PI3K/AKT signalling pathway inhibitor, which achieves a greater therapeutic efficacy than when either compound is used alone. The combination can be useful to produce tumor regression, to produce disease stability, to prevent or reduce metastasis, or other therapeutic endpoints, where the therapeutic effect is not observed when the agents are used alone, or where an enhanced effect is observed when the combination is administered.

The relative ratios of each compound in the combination can also be selected based on their respective mechanisms of action and the disease biology. For example, activating mutations of the B-RAF gene are observed in more than 60% of human melanomas and a composition for treatment of melanoma may advantageously comprise a formula I compound in a more potent amount than the compound which is a PI3K/AKT signalling pathway inhibitor. In comparison, where a cancer is associated with a mutation in the PI3K/AKT signalling pathway (e.g., ovarian and breast cancers), an agent which has activity in this signalling pathway can be present in more potent amounts relative to the Ref/MEK/ERK pathway inhibitor. The relative ratios of each compound can vary widely and this invention includes combinations for treating cancer where the amounts of the formula I compound and the second active agent can be adjusted routinely such that either is present in higher amounts.

The release of one or more agents of the combination can also be controlled, where appropriate, to provide the desired therapeutic activity when in a single dosage form, combination pack, kit or when in separate independent dosage forms.

### Assays

Activity of combinations of the present invention can be determined according to any effective in vitro or in vivo method.

### Kinase Activity

Kinase activity can be determined routinely using conventional assay methods. Kinase assays typically comprise the kinase enzyme, substrates, buffers, and components of a detection system. A typical kinase assay involves the reaction of a protein kinase with a peptide substrate and an ATP, such as ³²P-ATP, to produce a phosphorylated end-product (for instance, a phosphoprotein when a peptide substrate is used). The resulting end-product can be detected using any suitable method. When radioactive ATP is utilized, a radioactively labeled phosphoprotein can be separated from the unreacted gamma-³²P-ATP using an affinity membrane or gel electrophoresis, and then visualized on the gel using autoradiography or detected with a scintillation counter. Non-radioactive methods can also be used. Methods can utilize an antibody which recognizes the phosphorylated substrate, e.g., an anti-phosphotyrosine antibody. For instance, kinase enzyme can be incubated with a substrate in the presence of ATP and kinase buffer under conditions which are effective for the enzyme to phosphorylate the substrate. The reaction mixture can be separated, e.g., electrophoretically, and then phosphorylation of the substrate can be measured, e.g., by Western blotting using an anti-phosphotyrosine antibody. The antibody can be labeled with a detectable label, e.g., an enzyme, such as HRP, avidin or biotin, chemiluminescent reagents, etc. Other methods can utilize ELISA formats, affinity membrane separation, fluorescence polarization assays, luminescent assays, etc.

An alternative to a radioactive format is time-resolved fluorescence resonance energy transfer (TR-FRET). This method follows the standard kinase reaction, where a substrate, e.g., biotinylated poly(GluTyr), is phosphorylated by a protein kinase in the presence of ATP. The end-product can then detected with a europium chelate phosphospecific antibody (anti-phosphotyrosine or phosphoserine/threonine), and streptavidin-APC, which binds the biotinylated substrate. These two components are brought together spatially upon binding, and energy transfer from the phosphospecific antibody to the acceptor (SA-APC) produces fluorescent readout in the homogeneous format.

Raf/MEK/ERK activity

A c-Raf kinase assay can be performed with a c-Raf enzyme activated (phosphorylated) by Lck kinase. Lck-activated c-Raf (Lck/c-Raf) is produced in Sf9 insect cells by co-infecting cells with baculoviruses expressing, under the control of the polyhedrin promoter, GST-c-Raf (from amino acid 302 to amino acid 648) and Lck (full-length). Both baculoviruses are used at the multiplicity of infection of 2.5 and the cells are harvested 48 hours post infection.

MEK-1 protein is produced in Sf9 insect cells by infecting cells with the baculovirus expressing GST-MEK-1 (full-length) fusion protein at the multiplicity of infection of 5 and harvesting the cells 48 hours post infection. Similar purification procedure is used for GST-c-Raf 302-648 and GST-MEK-1.

Transfected cells are suspended at 100 mg of wet cell biomass per mL in a buffer containing 10 mM sodium phosphate, 140 mM sodium chloride pH 7.3, 0.5% Triton X-100 and the protease inhibitor cocktail. The cells are disrupted with a Polytron homogenizer and centrifuged 30,000 g for 30 minutes. The 30,000 g supernatant is applied applied onto GSH-Sepharose. The resin is washed with a buffer containing 50 mM Tris, pH 8.0, 150 mM NaCl and 0.01% Triton X-100. The GST-tagged proteins are eluted with a solution containing 100 mM Glutathione, 50 mM Tris, pH 8.0, 150 mM NaCl and 0.01% Triton X-100. The purified proteins are dialyzed into a buffer containing 20 mM Tris, pH 7.5, 150 mM NaCl and 20% Glycerol.

Test compounds are serially diluted in DMSO using three-fold dilutions to stock concentrations ranging typically from 50 μM to 20 nM (e.g., final concentrations in the assay can range from 1 μM to 0.4 nM). The c-Raf biochemical assay is performed as a radioactive filtermat assay in 96-well Costar polypropylene plates (Costar 3365). The plates are loaded with 75 μL solution containing 50 mM HEPES pH 7.5, 70 mM NaCl, 80 ng of Lck/c-Raf and 1 μg MEK-1. Subsequently, 2 μL of the serially diluted individual compounds is added to the reaction, prior to the addition of ATP. The reaction is initiated with 25 μL ATP solution containing 5 μM ATP and 0.3 μCi [33P]-ATP. The plates were sealed and incubated at 32° C. for 1 hour. The reaction is quenched with the addition of 50 μl of 4% Phosphoric Acid and harvested onto P30 filtermats (PerkinElmer) using a Wallac Tomtec Harvester. Filtermats are washed with 1% Phosphoric Acid first and deinonized H2O second. The filters are dried in a microwave, soaked in scintillation fluid and read in a Wallac 1205 Betaplate Counter (Wallac Inc., Atlanta, Ga., U.S.A.). The results are expressed as percent inhibition.

% Inhibition=[100−(Tib/Ti)]×100 where

Tib=(counts per minute with inhibitor)−(background)
Ti=(counts per minute without inhibitor)−(background)

Raf activity can also be monitored by its ability to initiate the cascade leading to ERK phosphorylation (i.e., raf/MEK/ERK), resulting in phospho-ERK. A Bio-Plex Phospho-ERK1/2 immunoassay can be performed as follows:

A 96-well phospho-ERK (pERK) immunoassay, using laser flow cytometry platform has been established to measure inhibition of basal pERK in cell lines. MDA-MB-231 cells are plated at 50,000 cells per well in 96-well microtitre plates in complete growth media. For effects of test compounds on basal pERK1/2 inhibition, the next day after plating, MDA-MB-231 cells are transferred to DMEM with 0.1% BSA and incubated with test compounds diluted 1:3 to a final concentration of 3 mM to 12 nM in 0.1% DMSO. Cells are incubated with test compounds for 2 h, washed, and lysed in Bio-Plex whole cell lysis buffer A. Samples are diluted with buffer B 1:1 (v/v) and directly transferred to assay plate or frozen at −80 C degrees until processed. 50 mL of diluted MDA-MB-231 cell lysates are incubated with about 2000 of 5 micron Bio-Plex beads conjugated with an anti-ERK1/2 antibody overnight on a shaker at room temperature. The next day, biotinylated phospho-ERK1/2 sandwich immunoassay is performed, beads are washed 3 times during each incubation and then 50 mL of PE-strepavidin is used as a developing reagent. The relative fluorescence units of pERK1/2 is detected by counting 25 beads with Bio-Plex flow cell (probe) at high sensitivity. The IC50 is calculated by taking untreated cells as maximum and no cells (beads only) as background.

### Phosphatidylinositol 3-Kinase Activity

PKI3 activity can be determined routinely, e.g., using commercially available kits (e.g., Perkin-Elmer, FlashPlate Platform), Frew et al., Anticancer Res., 14(6B):2425-8, 1994. See also, publications listed under PKI3 inhibitors.

### Akt Activity

AKT can be isolated from insect cells expressing His-tagged AKT1 (aa 136-480) as described in WO 05011700. Expressing cells are lysed in 25 mM HEPES, 100 mM NaCl, 20 mM imidazole; pH 7.5 using a polytron (5 mls lysis buffer/g cells). Cell debris is removed by centrifuging at 28,000×g for 30 minutes. The supernatant is filtered through a 4.5 micron filter then loaded onto a nickel-chelating column pre-equilibrated with lysis buffer. The column is washed with 5 column volumes (CV) of lysis buffer then with 5 CV of 20% buffer B, where buffer B is 25 mM HEPES, 100 mM NaCl, 300 mM imidazole; pH 7. His-tagged AKT1 (aa 136-480) is eluted with a 20-100% linear gradient of buffer B over 10 CV. His-tagged AKTI (136-480) eluting fractions are pooled and diluted three-fold with buffer C, where buffer C is 25 mM HEPES, pH 7. The sample is then chromatographed over a Q-Sepharose HP column pre-equilibrated with buffer C. The column is washed with 5 CV buffer C, then step eluted with 5 CV 10% D, 5 CV 20% D, 5 CV 30% D, 5 CV 50% D. and 5 CV of 100% D; where buffer D is 25 mM HEPES, 1000 mM NaCl; pH 7.5.

His-tagged AKTI (aa 136480) containing fractions are pooled and concentrated in a 10-kDa molecular weight cutoff concentrator. His-tagged AKT1 (aa 136480) is chromatographed over a Superdex 75 gel filtration column pre-equilibrated with 25 mM HEPES, 200 mM NaCl, 1 mM DTT; pH 7.5. His-tagged AKT1 (aa 136-480) fractions are examined using SDS-PAGE and mass spec. The protein is pooled, concentrated, and stored at 80° C.

His-tagged AKT2 (aa 138481) and His-tagged AKT3 (aa 135479) can be isolated and purified in a similar fashion.

AKT Enzyme Assay Compounds can be tested for AKT protein serine kinase inhibitory activity in substrate phosphorylation assays. This assay examines the ability of small molecule organic compounds to inhibit the serine phosphorylation of a peptide substrate. The substrate phosphorylation assays use the catalytic domains of AKT 1, 2, or 3. AKT 17 2 and 3 are also commercially available from Upstate USA, Inc. The method measures the ability of the isolated enzyme to catalyze the transfer of the gamma-phosphate from ATP onto the serine −72 residue of a biotinylated synthetic peptide (Biotin-ahx-ARKRERAYSFGHHA-amide). Substrate phosphorylation can be detected by the following procedure described in WO 05011700.

Assays are performed in 384 well U-bottom white plates. 10 nM activated AKT enzyme is incubated for 40 minutes at room temperature in an assay volume of 20 ul containing 50 mM MOPS, pH 7.5, 20 mM MgCl₂, 4 uM ATP, guM peptide, 0.04 uCi [g-³³P] ATP/well, 1 mM CHAPS, 2 mM DTT, and 1 μl of test compound in 100% DMSO. The reaction is stopped by the addition of 50 μl SPA bead mix (Dulbecco's PBS without Mg2+ and Ca2+, 0.1% Triton X-100, 5 mM EDTA, 50 μM ATP, 2.5 mg/ml Streptavidin-coated SPA beads). The plate is sealed, the beads are allowed to settle overnight, and then the plate was counted in a Packard Topcount Microplate

Scintillation Counter (Packard Instrument Co., Meriden, Conn.). The data for dose responses can be plotted as % Control calculated with the data reduction formula 100*(U1−C2)/(C1−C2) versus concentration of compound where U is the unknown value, C1 is the average control value obtained for DIVISO, and C2 is the average control value obtained for 0.1M EDTA. Data are fitted to the curve described by: y=((Vmax*x) K+x)) where Vmax is the upper asymptote and K is the IC50.

### Cell Proliferation

An example of a cell proliferation assay is described in the Examples below. However, proliferation assays can be performed by any suitable method. For example, a breast carcinoma cell proliferation assay can be performed as follows. Other cell types can be substituted for the MDA-MB-231 cell line.

Human breast carcinoma cells (MDA MB-231, NCI) are cultured in standard growth medium (DMEM) supplemented with 10% heat-inactivated FBS at 37° C. in 5% CO₂ (vol/vol) in a humidified incubator. Cells are plated at a density of 3000 cells per well in 90 μL growth medium in a 96 well culture dish. In order to determine T₀ₕ CTG values, 24 hours after plating, 100 μL of CellTiter-Glo Luminescent Reagent (Promega) is added to each well and incubated at room temperature for 30 minutes. Luminescence is recorded on a Wallac Victor II instrument. The CellTiter-Glo reagent results in cell lysis and generation of a luminescent signal proportional to the amount of ATP present, which, in turn is directly proportional to the number of cells present.

Test compounds are dissolved in 100% DMSO to prepare 10 mM stocks. Stocks are further diluted 1:400 in growth medium to yield working stocks of 25 uM test compound in 0.25% DMSO. Test compounds are serially diluted in growth medium containing 0.25% DMSO to maintain constant DMSO concentrations for all wells. 60 μL of diluted test compound are added to each culture well to give a final volume of 180 μL. The cells with and without individual test compounds are incubated for 72 hours at which time ATP dependent luminescence was measured, as described previously, to yield T₇₂ₕ values. Optionally, the IC₅₀ values can be determined with a least squares analysis program using compound concentration versus percent inhibition.

% Inhibition=[1−(T_{72h test}−T₀ₕ)/(T_{72h ctrl}−T₀ₕ)]×100, where

T_{72h test}=ATP dependent luminescence at 72 hours in the presence of test compound
T_{72h ctrl}=ATP dependent luminescence at 72 hours in the absence of test compound
T₀ₕ=ATP dependent luminescence at Time Zero.

### Angiogenesis

One useful model to study angiogenesis is based on the observation that, when a reconstituted basement membrane matrix, such as Matrigel, supplemented with growth factor (e.g., FGF-1), is injected subcutaneously into a host animal, endothelial cells are recruited into the matrix, forming new blood vessels over a period of several days. See, e.g., Passaniti et al., Lab. Invest., 67:519-528, 1992. By sampling the extract at different times, angiogenesis can be temporally dissected, permitting the identification of genes involved in all stages of angiogenesis, including, e.g., migration of endothelial cells into the matrix, commitment of endothelial cells to angiogenesis pathway, cell elongation and formation of sac-like spaces, and establishment of functional capillaries comprising connected, and linear structures containing red blood cells. To stabilize the growth factor and/or slow its release from the matrix, the growth factor can be bound to heparin or another stabilizing agent. The matrix can also be periodically re-infused with growth factor to enhance and extend the angiogenic process.

Other useful systems for studying angiogenesis, include, e.g., neovascularization of tumor explants (e.g., U.S. Pat. Nos. 5,192,744; 6,024,688), chicken chorioallantoic membrane (CAM) assay (e.g., Taylor and Folkman, Nature, 297:307-312, 1982; Eliceiri et al., J. Cell Biol., 140, 1255-1263, 1998), bovine capillary endothelial (BCE) cell assay (e.g., U.S. Pat. No. 6,024,688; Polyerini, P. J. et al., Methods Enzymol., 198: 440-450, 1991), migration assays, HUVEC (human umbilical cord vascular endothelial cell) growth inhibition assay (e.g., U.S. Pat. No. 6,060,449).

The present invention provides one or more of the following features:

A method of treating any of the aforementioned diseases and/or conditions, comprising administering effective amounts of at least one compound of formula I and at least one second compound which is a PI3K/AKT signalling pathway inhibitor.

A method of modulating (e.g., inhibiting) one or more aforementioned activities, comprising administering effective amounts of at least one compound of formula I and at least one second compound which is a PI3K/AKT signalling pathway inhibitor.

Combinations comprising at least one compound of formula I and at least one second compound which is a PI3K/AKT signalling pathway inhibitor.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever. The entire disclosure of all patents and publications, cited above and below are hereby incorporated by reference in their entirety.

### EXAMPLES

### Isolation and Culture of Human Cells

After obtaining informed consent, human fibroblasts were isolated from human foreskin following routine circumcision. The skin samples were stored at 4° C. in Hank's balanced salt solution without Ca2+ or Mg2+ (HBSS w/o Ca2+ or Mg2+) containing penicillin, gentamicin and amphotericin. The subcutaneous fat was trimmed off and the remaining cutis cut into pieces and digested in solution B containing 0.25% Trypsin as active ingredient (12) for approx. 19 h at 4° C. The action of the Trypsin was stopped with solution A (12), following which the epidermis was separated from the dermis. Human fibroblasts were obtained from dermal explants of human foreskin and cultured in Dulbecco's modified Eagle's medium (DMEM) with 10% fetal bovine serum (FBS). Fibroblasts up to passage 7 were used for melanoma reconstructs. Skmel28 (13) and 451Lu (14) metastatic human melanoma cells were cultured in RPMI 1640 medium supplemented with 10% FBS and in MCDB153/L15 medium containing 5 μg/ml insulin and 2% FBS, respectively (15).

### In Vitro Reconstruction of Metastatic Melanomas

The in vitro reconstruction of metastatic melanoma is based on the organotypic human skin culture technique (14). A cell-free buffered collagen solution was prepared consisting of rat tail collagen type I (BD Biosciences, Bedford, Mass., USA) at a final concentration of 1.35 mg/ml in Dulbecco's modified Eagle's medium with 10% FBS. 1.0 ml of the cell-free collagen solution was added to tissue culture inserts (Millicell PC, Millipore, Bedford, Mass., USA) placed in six-well tissue culture plates. While the acellular collagen layer was solidifying, a second collagen solution was prepared similar to the first with the addition of human fibroblasts and the melanoma cells SKMEL28 or 451 LU. Human fibroblasts and human melanoma cells from subconfluent cultures were trypsinised, washed and resuspended in the second collagen solution at a density of 15×10⁵/ml and a fibroblast to melanoma cell ratio of 1:1. 3.0 ml of the fibroblast and melanoma cell-containing collagen solution were placed over the solidified acellular collagen layer. After 5 days of incubation at 37° C., the fibroblast contraction force causes the collagen gel to contract. This structure represents the melanoma reconstruct in a dermal equivalent. For submerged culture conditions, 3 ml of melanoma cell culture medium supplemented with 10% FBS were added beneath the insert and 2 ml inside the insert to allow proliferation of the seeded cells. The culture medium was changed every two days. After 10 to 14 days of submerged culture, the melanoma reconstructs were harvested and evaluated.

### Treatment of Melanoma Cells with Signaling Pathway Inhibitors

For blockade of the AKT and MAPK signaling pathways, the PI3K inhibitor wortmannin (Sigma, Steinheim, Germany) and BAY 43-9006 alone or in combination were added directly to the culture medium of the melanoma reconstructs or melanoma cells in monolayer culture at 4 μM and 6 μM, respectively. These concentrations have been described previously to be effective for melanoma cells 6 (16). The culture medium was changed every two days. Melanoma reconstructs treated with culture medium or culture medium with the addition of DMSO served as controls. All experiments were done as duplicates and were repeated twice.

### Immunohistochemistry

Melanoma reconstructs were fixed with 4% formaldehyde for 8-9 h, dehydrated, and embedded in paraffin. Paraffin sections were stained with hematoxylin for routine light microscopy. For immunohistochemistry, paraffin sections of melanoma reconstructs were incubated with monoclonal antibodies for phospho-AKT (Ser473) and phospho-ERK (phospho-p44/42 MAP kinase, Thr202/Tyr204) (New England Biolabs, Frankfurt am Main, Germany), Ki-67 as a proliferation marker (Dianova, Hamburg, Germany), polyclonal antibodies for active caspase 3 (R&D Systems, Wiesbaden, Germany), or monoclonal antibodies for the β3 integrin subunit 17 and MelCAM (Novocastra Laboratories, Newcastle upon Tyne, UK). Sections were washed with PBS and incubated with the respective secondary antibody (Vector, Burlingame, Calif.) at room temperature for 30 min. After further washes with PBS the sections were incubated with the Vectastain® ABC-AP System (Vector, Burlingame, Calif.) at room temperature for 1 h. The sections were washed again with PBS, developed with neufuchsin and counterstained with hematoxylin.

### Proliferation Assay

Cells were seeded as triplicates in 96 well plates at a density of 1,500 cells per well in 150 μl medium (1×10⁴ cells per ml). The PI3K inhibitor wortmannin (Sigma, Steinheim, Germany) was directly added to the culture medium at concentrations ranging from 2-20 μM. BAY 43-9006 was added directly to the culture medium at concentrations ranging from 0.5-7 μM. Culture medium, cells treated with culture medium, and cells treated with culture medium with the addition of DMSO served as controls. Assay was started at timepoints indicated. Medium was discarded and each well was washed two times with PBS (without Ca2+ and Mg2+) and 100 μl of a solution containing 100 μg MUH (4-methylumbelliferyl-hepanoat)/ml PBS was added. Plates were incubated at 37° C. for one hour and measured in a Fluoroskan II (Labsystems, Helsinki), with an λₑₘ of 355 nm and an μₑₓ of 460 nm. The intensity of fluorescence indicates the number of vital cells in the wells (18,19).

### Flow Cytometry

Surface proteins of 1×10⁵-1×10⁶ 451Lu metastatic melanoma cells were stained as follows: Cells were pelleted for 5 min at 1,800 rpm (Heraeus variofuge 3.0R), blocked with 1×PBS/1% BSA, and after centrifugation incubated with antibodies against MeI-CAM (QBiogene-Alexis) or avβ3 integrin (BD Biosciences, Heidelberg) for 15 minutes at room temperature. Cells were washed with 1×PBS/1% BSA and subsequently incubated with anti mouse IgG-FITC (BD Biosciences, Heidelberg) or mouse IgG-isotype control-FITC alone (BD Biosciences, Heidelberg). After washing and pelleting the cells, the cell pellet was resuspended in 1×PBS and measured in a FACScalibur (BD Bioscienses, Heidelberg).

### Results

### Blockade of MAPK or AKT Signaling Pathways Induces Differential Effects on Melanoma Cell Growth and Adhesion Receptor Expression

To analyze the effects of inhibition of either the MAPK or AKT signaling pathways or both together on the proliferation of melanoma cells, we treated the metastatic melanoma cell line 451Lu in monolayer with the PI3K inhibitor wortmannin, BAY 43-9006, or both together. Based on previous studies (6-16) we chose 4 μM wortmannin and 6 μM BAY 43-9006 as the working concentrations.

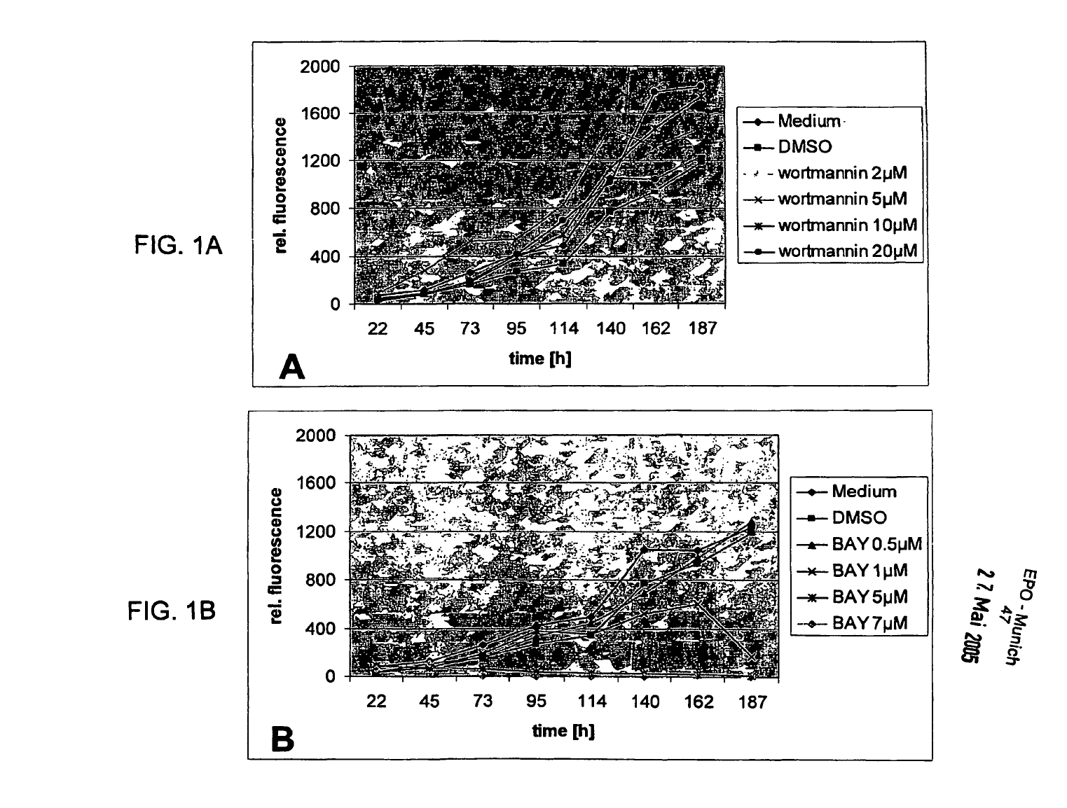

The effect of inhibition of these signaling pathways on cell proliferation was determined by a fluorimetric assay using 4-methylumbelliferyl heptanoate (MUH) (18, 19). Little or no effect on the number of proliferating cells was seen when comparing monolayer cultures from control 451Lu metastatic melanoma cells and 451Lu metastatic melanoma cells treated with the PI3K inhibitor wortmannin at dosages ranging from 2-20 μM (FIG. 1A). In contrast, the proliferation rate of 451Lu cells was significantly reduced after treatment with BAY 43-9006 at dosages ranging from 1-7 μM (FIG. 1B). Similar findings were obtained with Skmel28 metastatic melanoma cells. This indicates that blockade of MAPK but not of AKT signaling pathway inhibits melanoma cell proliferation in monolayer.

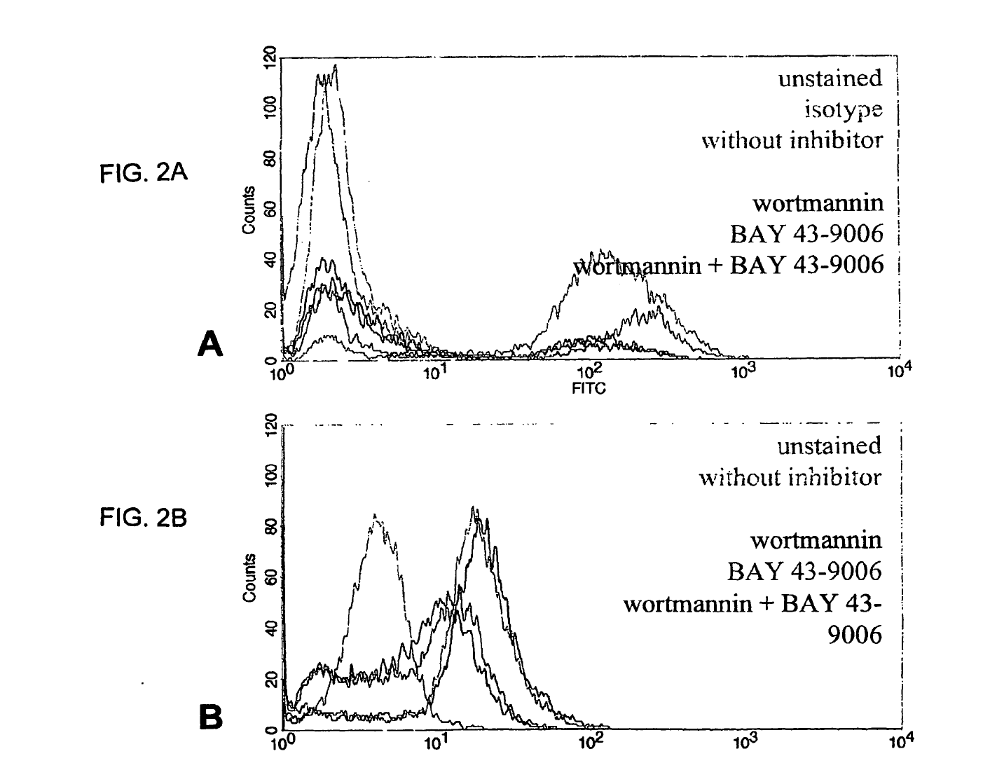

Furthermore, we examined whether inhibition of these signaling pathways affected expression of the adhesion molecules MelCAM and avβ3 known to play a key role in melanoma cell invasion. The effects of PI3K inhibitor wortmannin and BAY 43-9006 on MelCAM and avβ3 integrin expression in 451Lu metastatic melanoma cells were analyzed by flow cytometry 96 hours after beginning of treatment (FIG. 2). Interestingly, blockade of AKT downregulates the expression of the adhesion molecules MelCAM, but not avβ3 integrin, whereas blockade of ERK down-regulates the expression of avβ3 integrin, but not MelCAM. Similar effects were seen with Skmel28 metastatic melanoma cells.

### Blockade of MAPK but not of AKT Signaling Pathway Inhibits Proliferation of Melanoma Cells in a Human Dermal Reconstruct

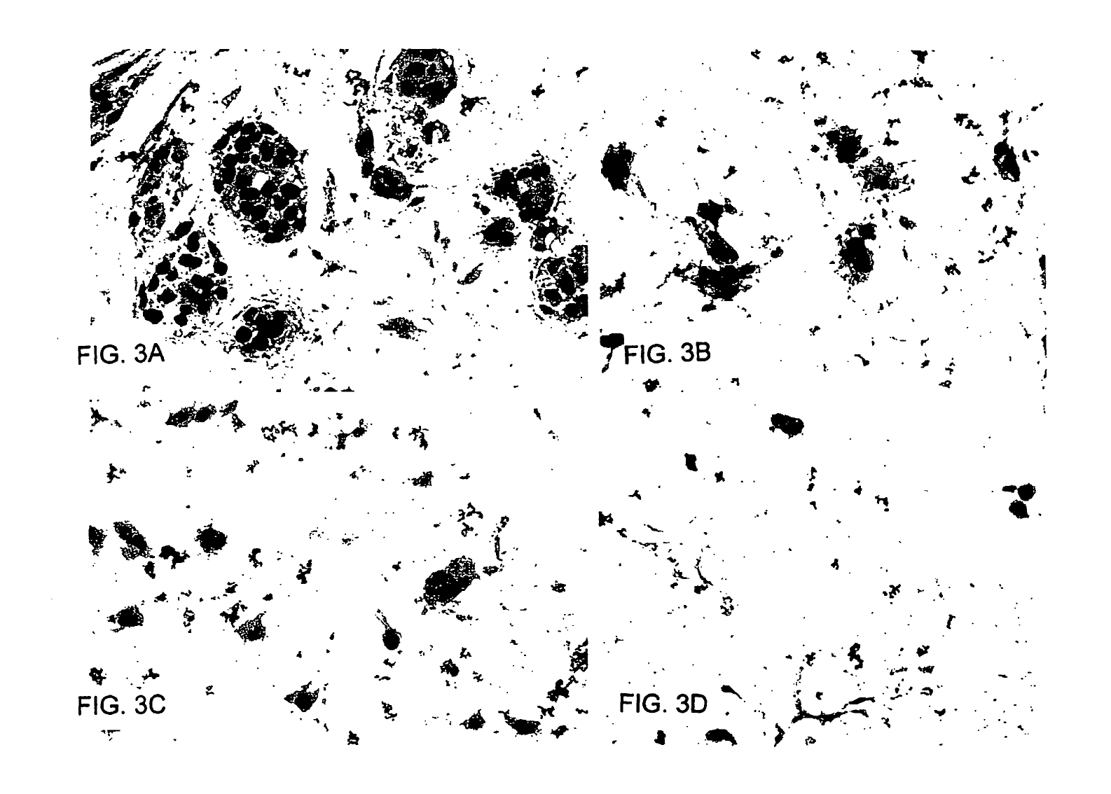

To determine whether the inhibition of the PI3K/AKT signaling pathway and the RAS/RAF/MEK/ERK signaling pathway is able to affect melanoma growth and survival in a physiological context, Skmel28 metastatic melanoma cells were incorporated into human dermal reconstructs. The reconstructed metastatic melanomas were treated with 4 μM wortmannin, 6 μM BAY 43-9006 or wortmannin combined with BAY 43-9006. The inhibitors were added to the culture medium every other day for 2-3 weeks. These inhibitor concentrations were effective in the inhibition of phosphorylation of either the AKT or MAP-kinase pathway as seen by immunohistochemistry for phosphorylated AKT or ERK, respectively. To evaluate the anti-proliferative effect of the inhibitors melanoma reconstructs were stained for Ki-67 proliferation marker. As can be seen in FIG. 3A most of the Skmel28 metastatic melanoma cells not treated with inhibitors or only with DMSO proliferated in the dermal reconstructs. Little or no effect on proliferation rate was observed in metastatic melanoma reconstructs treated with wortmannin (FIG. 3B). In contrast, treatment with BAY 43-9006 resulted in a significant decrease in cell proliferation (FIG. 3C). When Skmel28 metastatic melanoma cells were incorporated into dermal reconstructs and treated with the inhibitors in combination, proliferation of Skmel28 melanoma cells was completely blocked (FIG. 3D). These data indicate that BAY 43-9006 not only limits growth of metastatic melanoma cells in monolayer, but also in physiological context, and that the combined inhibition of PI3K and MAPK signaling pathways completely abrogates melanoma cell proliferation.

### Blockade of AKT and MAPK Signaling Pathways Induces Apoptosis of Melanoma Cells in Dermal Reconstructs

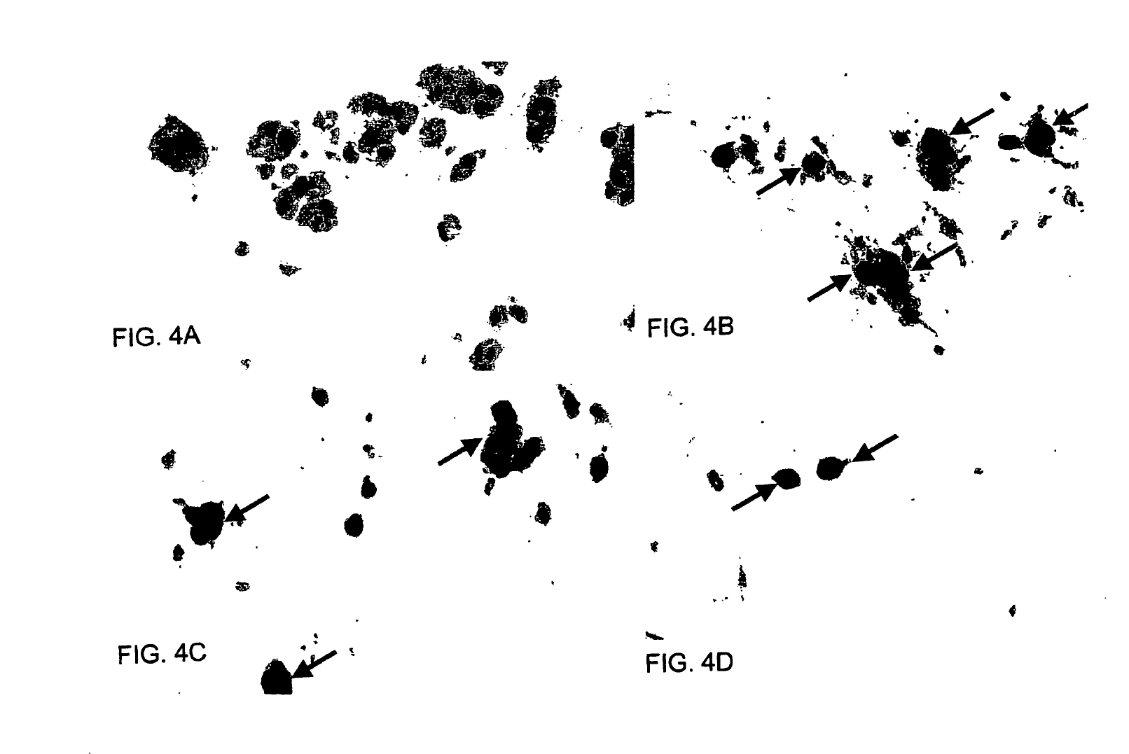

To investigate the effect of the PI3K inhibitor wortmannin and/or BAY 43-9006 on melanoma cell survival in organotypic culture, control and inhibitor-treated Skmel28 metastatic melanoma reconstructs were stained for active caspase 3 as a marker for ongoing apoptosis. Most of the control Skmel28 metastatic melanoma cells incorporated into dermal reconstructs were negative for active caspase 3 in the cytoplasm (FIG. 4A). In contrast, active caspase 3 was found in the majority of Skmel28 metastatic melanoma cells in human dermal reconstructs treated with the PI3K inhibitor wortmannin (FIG. 4B) or the BAY 43-9006 (FIG. 4C) or both (FIG. 4D). These observations suggest an involvement of both the AKT and MAPK signaling pathways in the survival of metastatic melanoma cells.

Blockade of AKT and MAPK Signaling Pathways Downregulates the Expression of the Adhesion Molecules MelCAM and avβ3 Integrin, Respectively, in Metastatic Melanoma Cells in Dermal Reconstructs

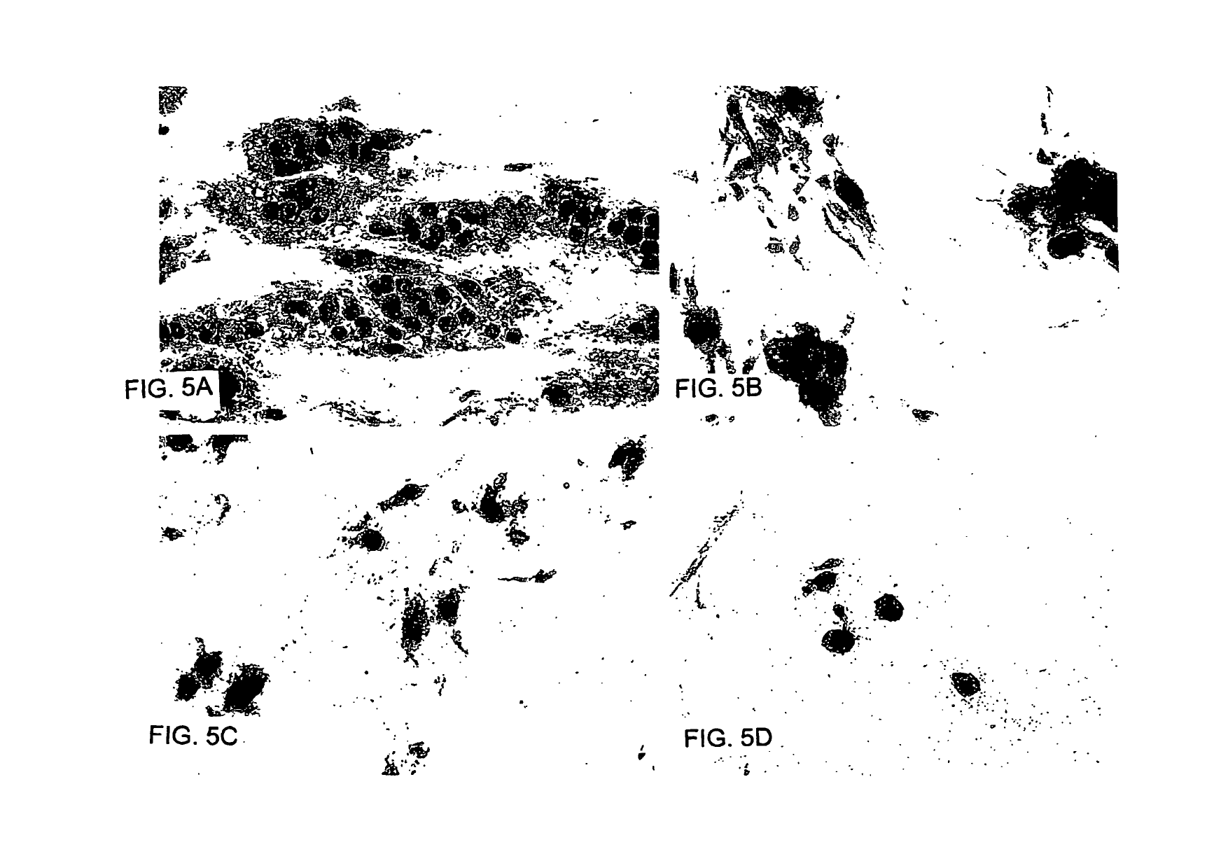

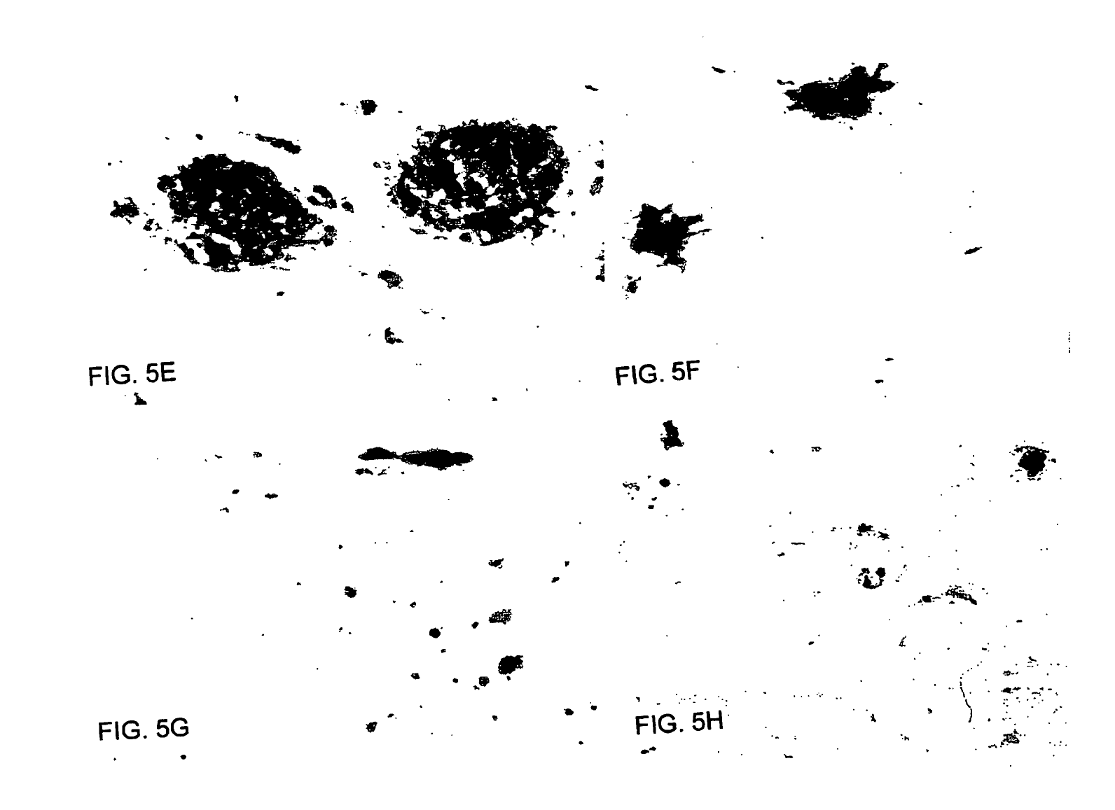

We and others described previously that the adhesion molecules MelCAM and avβ3 integrin play key roles in melanoma progression and invasion (9, 6, 10, 11). Therefore, we examined the effects of blocking AKT and RAF on MelCAM and avβ3 integrin expression. For the analysis of receptor expression in a physiological context, the metastatic melanoma cells Skmel28 and WM451Lu were incorporated into human dermal reconstructs and treated with the PI3K inhibitor wortmannin (4 μmM), BAY 43-9006 (6 μM) or a combination of both inhibitors. Control and inhibitor-treated metastatic melanoma reconstructs were stained for the adhesion molecules MelCAM (FIG. 5A-D) and β3 integrin (FIG. 5E-H), respectively.

Wortmannin treatment downregulated the expression of MelCAM while the expression of β3 integrin was not affected (FIG. 5A-H). On the other hand, β3 integrin expression was significantly reduced by BAY 43-9006 treatment, whereas MelCAM expression was not affected. Treatment with both inhibitors resulted in downregulation of MelCAM and β3 integrin expression. These data indicate that both signaling pathways—PI3K/AKT and MAP-kinase—have to be blocked to downregulate both adhesion molecules.

### Blockade of AKT and MAPK Signaling Pathways Inhibits Invasive Melanoma Growth

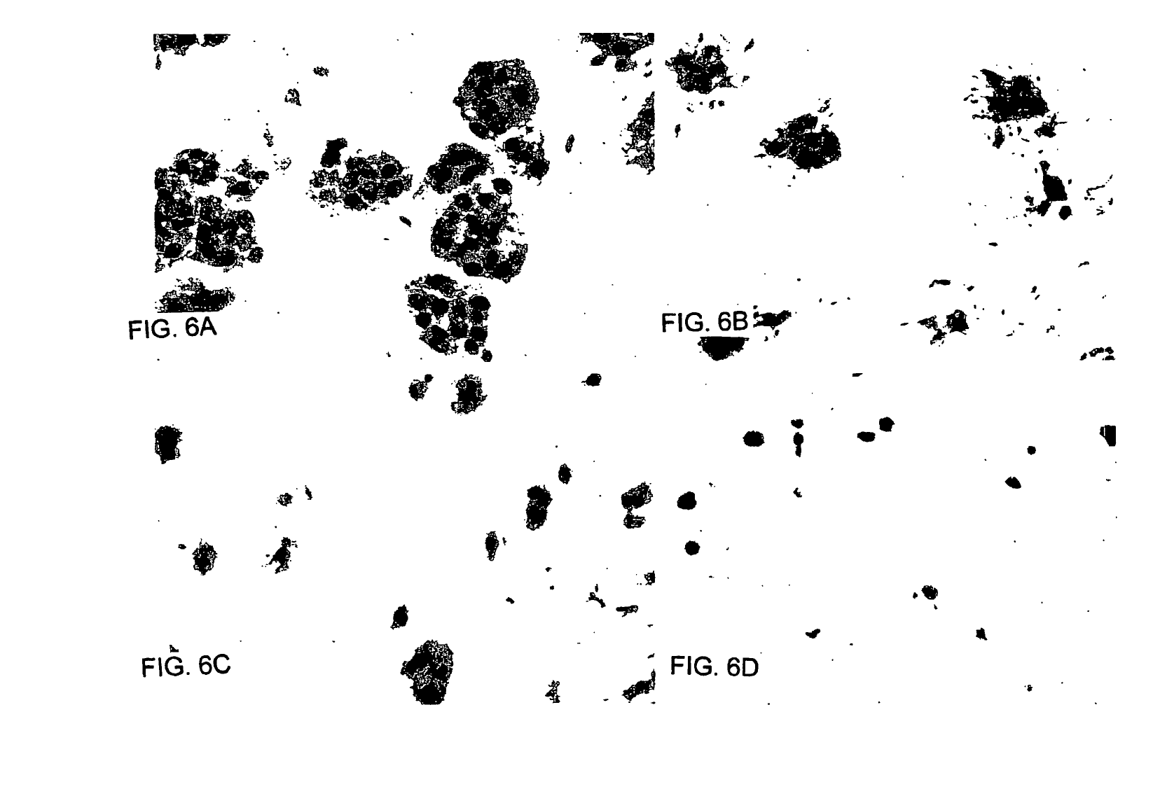

Finally, we determined whether the inhibition of the PI3K/AKT signaling pathway and the MAP kinase signaling pathway is able to affect invasive melanoma growth in a physiological context. Skmel28 metastatic melanoma cells were incorporated into human dermal reconstructs. The reconstructed metastatic melanomas were treated with 4 μM wortmannin, 6 μM BAY 43-9006, or wortmannin combined with BAY 43-9006. When metastatic Skmel28 melanoma cells were incorporated into reconstructed human dermis they exhibited rapid growth of multiple tumor cell nests throughout the entire dermis (FIG. 6A). Either the inhibition of the AKT signaling pathway by wortmannin or the inhibition of the MAPK signaling pathway, etc., by BAY 43-9006 resulted in a reduced invasive tumor growth of Skmel28 metastatic melanoma cells in reconstructed dermis (FIGS. 6B and C). After treatment with the PI3K inhibitor wortmannin (FIG. 6B) melanoma cell nests were reduced in number and size and appeared to be loosened suggesting a decrease in melanoma—melanoma cell adhesion. Furthermore, melanoma cells displayed a multidendritic morphology. The application of inhibitor BAY 43-9006 (FIG. 6C) also reduced the number and size of melanoma cell nests.

Small melanoma cell nests and single melanoma cells were scattered throughout the dermis. Moreover, simultaneous blockade of the AKT and MAPK signaling pathways by wortmannin combined with BAY 43-9006 completely abrogated invasive melanoma growth with very few rounded melanoma cells left in the dermis (FIG. 6D). Similar results were obtained with the metastatic melanoma cell line 451Lu.

### REFERENCES

1. Blume-Jensen, and Hunter, Nature, 411: 355-365, 2001.
2. Davies et al., Nature, 417: 949-954, 2002.
3. Satyamoorthy et al., Cancer Res, 63: 756-759, 2003.
4. Ahmad et al., The Royal Marsden experience. 2004.
5. Nicholson and Anderson, Cell Signal, 14: 381-395, 2002.
6. Li et al., Oncogene, 22: 6891-6899, 2003.
7. Vivanco and Sawyers, Nat Rev Cancer, 2: 489-501, 2002.
8. Downward, Curr Opin Cell Biol, 10: 262-267, 1998.
9. Satyamoorthy et al. Oncogene, 20: 4676-4684, 2001.
10. Hsu et al., Am J Pathol, 153: 1435-1442, 1998.
11. Sturm et al., Cancer Res, 62: 226-232, 2002.
12. Pittelkow et al., Mayo Clin Proc, 61: 771-777, 1986.
13. Carey et al., Proc. Natl. Acad. Sci. U.S.A, 73: 3278-3282, 1976.
14. Meie et al., Am J Pathol, 156: 193-200, 2000.
15. Hsu et al., The Wistar melanoma (WM) cell lines. In J. R. W. Masters and B. Palsson (eds.), Human Cell. Culture, pp. 259-274. Norwell, Mass.: Kluwer Academic Publishers, 1999.
16. Karasarides et al., Oncogene, 23: 6292-6298, 2004.
17. Van Belle et al., Hum Pathol, 30: 562-567, 1999.
18. Virag et al., J. Immunol. Methods, 185: 199-208, 1995.
19. Zouboulis et al., Melanoma Res., 1: 91-95, 1991.

1. A combination comprising at least one compound of formula I or a pharmaceutically acceptable salt, polymorph, solvate, hydrate, metabolite, prodrug or diastereoisomeric form thereof,
wherein said compound of formula I is: 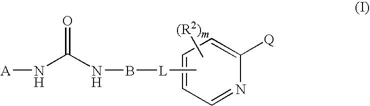
wherein
Q is —C(O)Rₓ
Rₐ is hydroxy, C₁₋₄ alkyl, C₁₋₄ alkoxy or NRₐR_{b},
Rₐ and R_{b} are independently:
a) hydrogen;
b) C₁₋₄ alkyl, optionally substituted by
hydroxy,
—C₁₋₄ alkoxy,
a heteroaryl group selected from pyrrole, furan, thiophene, imidazole, pyrazole, thiazole, oxazole, isoxazole, isothiazole, triazole, tetrazole, thiadiazole, oxadiazole, pyridine, pyrimidine, pyridazine, pyrazine, triazine, benzoxazole, isoquioline, quinolines and imidazopyrimidine
a heterocyclic group selected from tetrahydropyran, tetrahydrofuran, 1,3-dioxolane, 1,4-dioxane, morpholine, thiomorpholine, piperazine, piperidine, piperidinone, tetrahydropyrimidone, pentamethylene sulfide, tetramethylene sulfide, dihydropyrane, dihydrofuran, and dihydrothiophene,
amino, —NH₂, optionally substituted by one or two C₁₋₄ alkyl groups, or
phenyl,
c) phenyl optionally substituted with
halogen, or
amino, —NH₂, optionally substituted by one or two C₁₋₄ alkyl, or
d)—a heteroaryl group selected from pyrrole, furan, thiophene, imidazole, pyrazole, thiazole, oxazole, isoxazole, isothiazole, triazole, tetrazole, thiadiazole, oxadiazole, pyridine, pyrimidine, pyridazine, pyrazine, triazine, benzoxazole, isoquioline, quinoline and imidazopyrimidine;
A is an optionally substituted phenyl group of formula 1xx: 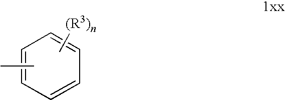
an optionally substituted pyridinyl group of formula 1x: 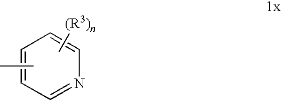
or an optionally substituted naphthyl moiety of formula 1y: 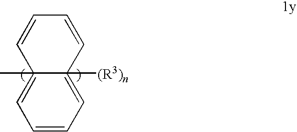
B is optionally substituted phenyl or naphthyl of formulas 2a and 2b: 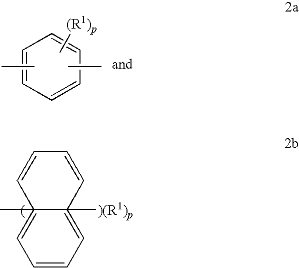
L is a bridging group which is —S— or —O—,
p is 0, 1, 2, 3, or 4,
n is 0, 1, 2, 3, 4, 5 or 6,
m is 0, 1, 2 or 3,
each R¹ is independently: halogen, C₁₋₅ haloalkyl, NO₂, C(O)NR⁴R⁵, C₁₋₆ alkyl, C₁₋₆ dialkylamine, C₁₋₃ alkylamine, CN, amino, hydroxy or C₁₋₃ alkoxy.
each R² is independently: C₁₋₅ alkyl, C₁₋₅ haloalkyl, C₁₋₃ alkoxy, N-oxo or N-hydroxy,
each R³ is independently: halogen, R⁴, OR⁴, S(O)R⁴, C(O)R⁴, C(O)NR⁴R⁵, oxo, cyano or nitro (NO₂) and
R⁴ and R⁵ are independently hydrogen, C₁₋₆ alkyl, or up to per-halogenated C₁₋₆ alkyl.
and at least one second compound which is an PI3K/AKT signalling pathway inhibitor.

2. The combination as in claim 1 wherein
A is 3-tert butyl phenyl, 5-tert butyl-2-methoxyphenyl, 5-(trifluoromethyl)-2 phenyl, 3-(trifluoromethyl)-4 chlorophenyl, 3-(trifluoromethyl)-4-bromophenyl or 5-(trifluoromethyl)-4-chloro-2 methoxyphenyl;
B is 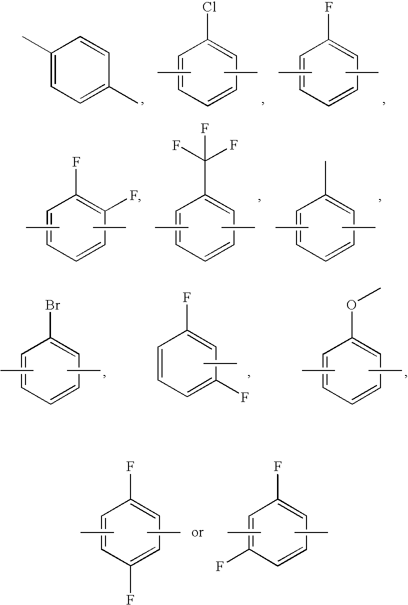
R¹ is fluorine, chorine, bromine, methyl, NO₂, C(O)NH₂, methoxy, SCH₃, trifluoromethyl, or methanesulfonyl;
R² is methyl, ethyl, propyl, oxygen, or cyano and
R³ is trifluoromethyl, methyl, ethyl, propyl, butyl, isopropyl, tert-butyl, chlorine, fluorine, bromine, cyano, methoxy, acetyl, trifluoromethanesulfonyl, trifluoromethoxy, or trifluoromethylthio.

3. The combination as in claim 1, wherein the compound of formula I is also of formula II below or salts, polymorphs, solvates, hydrates, metabolites, prodrugs or diastereoisomeric forms thereof: 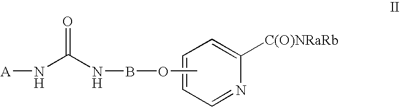
wherein
Ra and Rb are independently hydrogen and C₁-C₄ alkyl,
B of formula II is 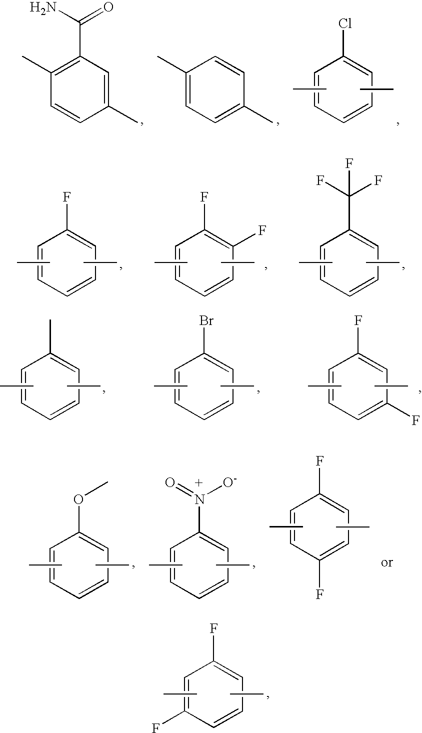
wherein the urea group, —NH—C(O)—NH—, and the oxygen bridging group are not bound to contiguous ring carbons of B, but rather have 1 or 2 ring carbons separating them, and
A of formula (II) is 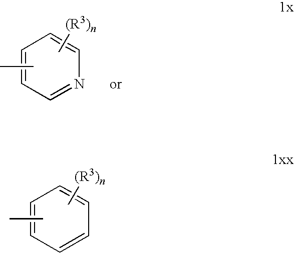
wherein the variable n is 0, 1, 2, 3 or 4, and
R³ is trifluoromethyl, methyl, ethyl, propyl, butyl, isopropyl, tert-butyl, chlorine, fluorine, bromine, cyano, methoxy, acetyl, trifluoromethanesulfonyl, trifluoromethoxy, or trifluoromethylthio.

4. The combination of claim 2 wherein, each R³ substituent is chlorine, trifluoromethyl, tert-butyl or methoxy,
A of formula II is 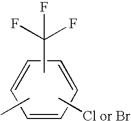
and
B of formula II is phenylene, fluoro substituted phenylene or difluoro substituted phenylene.

5. The combination of claim 1 wherein the compound of formula I is also of formula X below or salts, polymorphs, solvates, hydrates, metabolites, prodrugs or diastereoisomeric forms thereof: 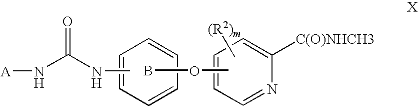
wherein phenyl ring “B” optionally has one halogen substituent,
A is an optionally substituted phenyl group of formula 1xx: 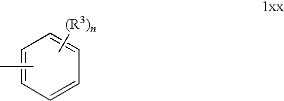
an optionally substituted pyridinyl group of formula 1x: 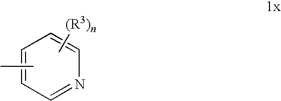
or an optionally substituted naphthyl moiety of formula 1y: 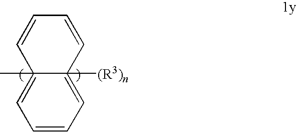
n is 0, 1, 2, 3, 4, 5 or 6,
m is 0, 1, 2 or 3,
each R² is independently: C₁₋₅ alkyl, C₁₋₅ haloalkyl, C₁₋₃ alkoxy, N-oxo or N-hydroxy,
each R³ is independently: halogen, R⁴, OR⁴, S(O)R⁴, C(O)R⁴, C(O)NR⁴R⁵, oxo, cyano or nitro (NO₂) and
R⁴ and R⁵ are independently hydrogen, C₁₋₆ alkyl, or up to per-halogenated C₁₋₆ alkyl.

6. The combination as in claim 5 wherein m is zero and A is substituted phenyl with at least one substituent, R³.

7. The combination as in claim 6 wherein R³ is halogen, trifluoromethyl and/or methoxy.

8. The combination of claim 1 wherein the compound of formula I also has the structure of one of formulas Z1 or Z2 below or a salt, polymorph, solvate, hydrate, metabolite, prodrug or diastereoisomeric form thereof:

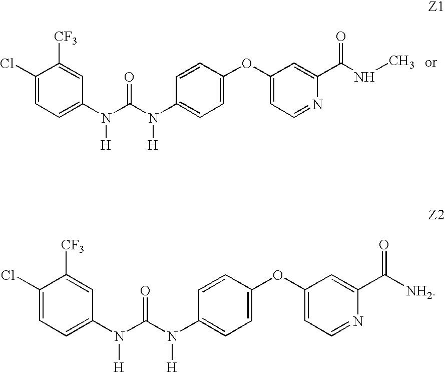

9. The combination of claim 8 wherein the compound of formula I is the tosylate salt of the compound of formula Z1.

10. The combination of claim 1, wherein the PI3K/AKT signalling pathway inhibitor is selected from the group of compounds consisting of FTY720, UCN-01, celecoxib and analogs thereof, 3-deoxy-D-myo-inositol analogs, 2′-substituted, 3′-deoxy-phosphatidyl-myo-inositol analogs, 3-(imidazo[1,2-a]pyridin-3-yl) derivatives, Ly294002, quinazoline-4-one derivatives, 3-(hetero)aryloxy substituted benzo(b)thiophene derivatives, viridins, semi-synthetic viridins, Akt-1-1, Akt-1-1,2, API-59CJ-Ome, 1-H-imidazo[4,5-c]pyridinyl compounds, indole-3-carbinol and derivatives thereof, perifosine, phosphatidylinositol ether lipid analogs, triciribine and FKBP12 enhancer.

11. The combination of claim 10, wherein the celecoxib analogs are OSU-03012, OSU-03013.

12. The combination of claim 10, wherein the 3-deoxy-D-myo-inositol analog is PX-316.

13. The combination of claim 10, wherein the quinazoline-4-one derivative is IC486068.

14. The combination of claim 10, wherein the semi-synthetic viridian is PX-866.

15. The combination of claim 10, wherein said second compound is an FKBP12 enhancer.

16. The combination of claim 1 wherein the PI3K/AKT signalling pathway inhibitor is celecoxib, OSU-03012, OSU-03013, PX-316, 2′-substituted, 3′-deoxy-phosphatidyl-myo-inositol derivatives, 3-(imidazo[1,2-a]pyridin-3-yl) derivatives, Ly294002, IC486068, 3-(hetero)aryloxy substituted benzo(b)thiophene derivatives, PX-866, perifosine, triciribine, FKBP12 enhancer, phosphatidylinositol ether lipid analogues, wortmannin or rapamycin or derivatives thereof, or a pharmaceutically-acceptable salt thereof.

17. The combination of claim 1, wherein said second compound is a wortmannin compound of
formula W: 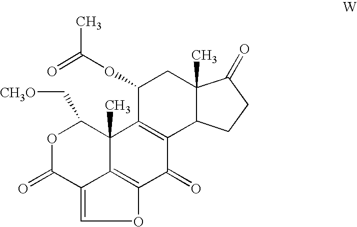
a derivative or analog of a wortmannin compound of formula W, a pharmaceutically acceptable salt of the wortmannin compound of formula W, or a pharmaceutically acceptable salt of the derivative or analog of the wortmannin compound of formula W.

18. The combination of claim 17, wherein said derivative or analog of the formula W
is selected from
a) compounds of formula W1 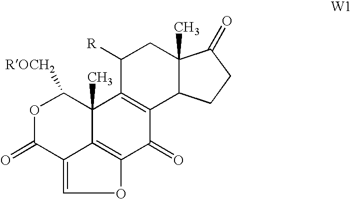
where R is H (11-desacetoxywortmannin) or acetoxy and R′ is C₁-C₆alkyl,
b) Δ9,11-dehydrodesacetoxywortmannin compounds of formula W2 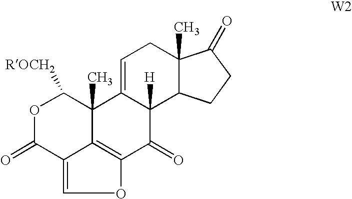
where R′ is C₁-C₆ alkyl,
c) 17(α-dihydro-wortmannin compounds of formula W3 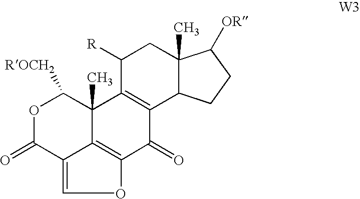
where R is H or acetoxy and R′ is C₁-C₆ alkyl and R″ is H, C₁-C₆ alkyl, —C(O)OH or —C(O)O—C₁-C₆ alkyl;
d) open A-ring acid or ester of wortmannin compounds of formula W4 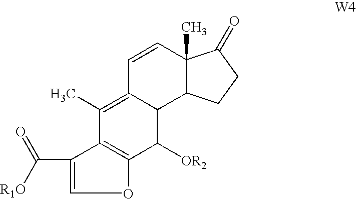
where R₁ is H, methyl or ethyl and R₂ is H or methyl or
e) 11-substituted and 17-substituted derivatives of wortmannin of formula W5 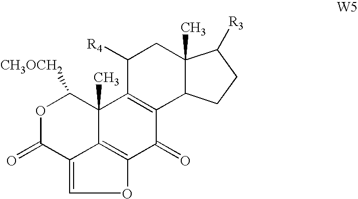
where R₄ is ═O or —O(CO)R₆, R₃ is ═O, —OH or —O(CO)R₆, each R₆ is independently phenyl, C₁-C₆ alkyl or substituted C₁-C₆ alkyl, where R₄ is ═O or —OH, R₃ is not ═O.

19. The combination of claim 1, wherein said second compound is an Akt-kinase inhibitor.

20. The combination of claim 1, wherein said second compound is Akt-1-1, Akt-1-1,2, API-59CJ-Ome, 1-H-imidazo[4,5-c]pyridinyl derivatives, indole-3-carbinol and derivatives thereof, perifosine, phosphatidylinositol ether lipid analogues, triciribine, or a pharmaceutically-acceptable salt thereof.

21. The combination of claim 1, wherein said second compound is an mTOR inhibitor.

22. The combination of claim 1, wherein said second compound is rapamycin, temsirolimus, everolimus, AP23573, AP23675, AP23464, AP23841, 40-(2-hydroxyethyl)rapamycin, 40-[3-hydroxy(hydroxymethyl)methylpropanoate]-rapamycin, 40-epi-(tetrazolyt)-rapamycin, 32-deoxorapamycin, or 16-pentynyloxy-32(S)-dihydrorapamycin, SAR 943 or a pharmaceutically-acceptable salt thereof.

23. The combination of claim 1 comprising a compound of formula (I) and wortmannin.

24. The combination of claim 1 comprising a compound of formula (I) and rapamycin.

25. The combination of claim 1, wherein said second compound is a PI3-kinase inhibitor.

26. The combination of claim 1, wherein said second compound is celecoxib, OSU-03012, OSU-03013, PX-316, 2′-substituted 3′-deoxy-phosphatidyl-myo-inositol derivatives, 3-(imidazo[1,2-a]pyridin-3-yl) derivatives, Ly294002, IC486068, 3-(hetero)aryloxy substituted benzo(b)thiophene derivatives, PX-866 or a pharmaceutically-acceptable salts thereof.

27. The combination of claim 1 wherein the amounts of the active ingredients of the combination are synergistic.

28. The combination of claim 1 for treating cancer.

29. The combination of claim 28, wherein said cancer is melanoma, hepatocellular cancer, renal cell carcinoma, non small lung cancer, ovarian cancer, prostate cancer, colorectal cancer, breast cancer or pancreatic cancer.

30. A method for treating cancer in a subject in need thereof comprising administering effective amounts of at least one compound of formula I or a pharmaceutically acceptable salt, polymorph, solvate, hydrate, metabolite, prodrug or diastereoisomeric form thereof
wherein said compound of formula I is: 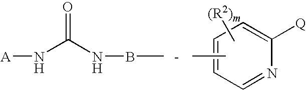
wherein
Q is —C(O)Rₓ,
Rₓ is hydroxy, C₁₋₄ alkyl, C₁₋₄ alkoxy or NRₐR_{b},
Rₐ and R_{b} are independently:
a) hydrogen;
b) C₁₋₄ alkyl, optionally substituted by
hydroxy,
C₁₋₄ alkoxy,
a heteroaryl group selected from pyrrole, furan, thiophene, imidazole, pyrazole, thiazole, oxazole, isoxazole, isothiazole, triazole, tetrazole, thiadiazole, oxadiazole, pyridine, pyrimidine, pyridazine, pyrazine, triazine, benzoxazole, isoquioline, quinolines and imidazopyrimidine
a heterocyclic group selected from tetrahydropyran, tetrahydrofuran, 1,3-dioxolane, 1,4-dioxane, morpholine, thiomorpholine, piperazine, piperidine, piperidinone, tetrahydropyrimidone, pentamethylene sulfide, tetramethylene sulfide, dihydropyrane, dihydrofuran, and dihydrothiophene,
amino, —NH₂, optionally substituted by one or two C₁₋₄ alkyl groups, or
phenyl,
c) phenyl optionally substituted with
halogen, or
amino, —NH₂, optionally substituted by one or two C₁₋₄ alkyl, or
d)—a heteroaryl group selected from pyrrole, furan, thiophene, imidazole, pyrazole, thiazole, oxazole, isoxazole, isothiazole, triazole, tetrazole, thiadiazole, oxadiazole, pyridine, pyrimidine, pyridazine, pyrazine, triazine, benzoxazole, isoquioline, quinoline and imidazopyrimidine;
A is an optionally substituted phenyl group of formula 1xx: 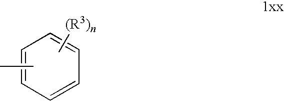
an optionally substituted pyridinyl group of formula 1x: 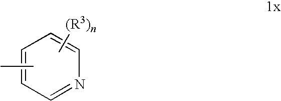
or an optionally substituted naphthyl moiety of formula 1y: 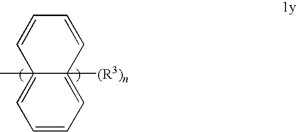
B is optionally substituted phenyl or naphthyl of formulas 2a and 2b: 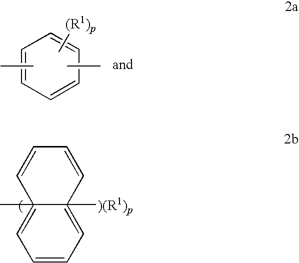
L is a bridging group which is —S— or —O—,
p is 0, 1, 2, 3, or 4,
n is 0, 1, 2, 3, 4, 5 or 6,
m is 0, 1, 2 or 3,
each R¹ is independently: halogen, C₁₋₅ haloalkyl, NO₂, C(O)NR⁴R⁵, C₁₋₆ alkyl, C₁₋₆ dialkylamine, C₁₋₃ alkylamine, CN, amino, hydroxy or C₁₋₃ alkoxy.
each R² is independently: C₁₋₅ alkyl, C₁₋₅ haloalkyl, C₁₋₃ alkoxy, N-oxo or N-hydroxy,
each R³ is independently: halogen, R⁴, OR⁴, S(O)R⁴, C(O)R⁴, C(O)NR⁴R⁵, oxo, cyano or nitro (NO₂) and
R⁴ and R⁵ are independently hydrogen, C₁₋₆ alkyl, or up to per-halogenated C₁₋₆ alkyl;
and at least one second compound as defined in any of claims 1 to 26.

31. A process for manufacturing a combination of claim 1 for treating cancer.

32. The process of claim 31, wherein said cancer is melanoma, hepatocellular cancer, renal cell carcinoma, non small lung cancer, ovarian cancer, prostate cancer, colorectal cancer, breast cancer or pancreatic cancer.

33. A pharmaceutical composition comprising a therapeutically effective amount of a combination of claim 1 and a pharmaceutically acceptable excipient.
